(19) United States
(12) Patent Application Publication
(10) Pub. No.: US 2007/0269422 A1
(43) Pub. Date: Nov. 22, 2007

(54) Serum albumin binding proteins with long half-lives

(75) Inventors: Els Beirnaert, Bellem (BE); Hilde Adi Pierrette Revets, Meise (BE); Hendricus Renerus Jacobus Mattheus Hoogenboom, Maastricht (NL); Heidi Maria Florence Jonckheere, Heverlee (BE); Torsten Dreier, Sint-Martens-Latem (BE)

Correspondence Address: WOLF GREENFIELD & SACKS, P.C., 600 ATLANTIC AVENUE, BOSTON, MA, 02210-2206, US

(73) Assignee: Ablynx N.V., Ghent-Zwijnaarde (BE)

(21) Appl. No.: 11/517,816

(22) Filed: Sep. 8, 2006

Related U.S. Application Data
(63) Continuation-in-part of application No. PCT/EP06/04679, filed on May 17, 2006.

(51) Int. Cl. A61K 39/395; A61K 31/7052; A61K 48/00; C07H 21/04; C07K 14/00; C07K 16/18; C12N 5/06; C12P 1/04

(52) U.S. Cl. 424/130.1; 435/325; 435/69.1; 514/2; 514/44; 530/324; 530/387.1; 530/387.3; 536/23.1

(57) ABSTRACT

The present invention relates to amino acid sequences that are capable of binding to serum albumin; to compounds, proteins and polypeptides comprising or essentially consisting of such amino acid sequences; to nucleic acids that encode such amino acid sequences, proteins or polypeptides; to compositions, and in particular pharmaceutical compositions, that comprise such amino acid sequences, proteins and polypeptides; and to uses of such amino acid sequences, proteins and polypeptides. Particularly, the amino acid sequences and compounds of the present invention bind to or otherwise associate with serum albumin in such a way that, when the amino acid sequence or compound is bound to or otherwise associated with a serum albumin molecule in a primate, it exhibits a serum half-life of at least 50% of the natural half-life of serum albumin in said primate.

### RELATED APPLICATIONS

This application is a continuation-in-part of International Application PCT/EP2006/004679 designating the United States of America, filed May 17, 2006, and claims the benefit under 35 U.S.C. § 119(e) of U.S. provisional application Ser. No. 60/682,332, filed May 18, 2005, the entire disclosures of which are incorporated herein by reference.

### FIELD OF THE INVENTION

The present invention relates to amino acid sequences that are capable of binding to serum albumin; to compounds, proteins and polypeptides comprising or essentially consisting of such amino acid sequences; to nucleic acids that encode such amino acid sequences, proteins or polypeptides; to compositions, and in particular pharmaceutical compositions, that comprise such amino acid sequences, proteins and polypeptides; and to uses of such amino acid sequences, proteins and polypeptides. Particularly, the amino acid sequences and compounds of the present invention bind to or otherwise associate with serum albumin in such a way that, when the amino acid sequence or compound is bound to or otherwise associated with a serum albumin molecule in a primate, it exhibits a serum half-life of at least 50% of the natural half-life of serum albumin in said primate.

Other aspects, embodiments, advantages and applications of the invention will become clear from the further description herein.

### BACKGROUND OF THE INVENTION

Amino acid sequences that are capable of binding to human serum albumin and uses thereof in polypeptide constructs in order to increase the half-life of therapeutically relevant proteins and polypeptides are known in the art.

For example, WO 91/01743, WO 01/45746 and WO 02/076489 describe peptide moieties binding to serum albumin that can be fused to therapeutic proteins and other therapeutic compounds and entities in order to increase the half-life thereof. However, these peptide moieties are of bacterial or synthetic origin, which is less preferred for use in therapeutics.

WO 04/041865 by Ablynx N. V. describes Nanobodies® directed against serum albumin (and in particular against human serum albumin) that can be linked to other proteins (such as one or more other Nanobodies® directed against a desired target) in order to increase the half-life of said protein.

The neonatal Fc receptor (FcRn), also termed “Brambell receptor”, is involved in prolonging the life-span of albumin in circulation (see Chaudhury et al., The Journal of Experimental Medicine, vol. 3, no. 197, 315-322 (2003)). The FcRn receptor is an integral membrane glycoprotein consisting of a soluble light chain consisting of β2-microglobulin, noncovalently bound to a 43 kD α chain with three extracellular domains, a transmembrane region and a cytoplasmic tail of about 50 amino acids. The cytoplasmic tail contains a dinucleotide motif-based endocytosis signal implicated in the internalization of the receptor. The α chain is a member of the nonclassical MHC I family of proteins. The β2m association with the α chain is critical for correct folding of FcRn and exiting the endoplasmic reticulum for routing to endosomes and the cell surface.

The overall structure of FcRn is similar to that of class I molecules. The α-1 and α-2 regions resemble a platform composed of eight antiparallel β strands forming a single β-sheet topped by two antiparallel α-helices very closely resembling the peptide cleft in MHC I molecules. Owing to an overall repositioning of the α-1 helix and bending of the C-terminal portion of the α-2 helix due to a break in the helix introduced by the presence of Pro162, the FcRn helices are considerably closer together, occluding peptide binding. The side chain of Arg164 of FcRn also occludes the potential interaction of the peptide N-terminus with the MHC pocket. Further, salt bridge and hydrophobic interaction between the α-1 and α-2 helices may also contribute to the groove closure.

FcRn therefore, does not participate in antigen presentation, and the peptide cleft is empty.

FcRn binds and transports IgG across the placental syncytiotrophoblast from maternal circulation to fetal circulation and protects IgG from degradation in adults. In addition to homeostasis, FcRn controls transcytosis of IgG in tissues. FcRn is localized in epithelial cells, endothelial cells and hepatocytes.

According to Chaudhury et al. (supra), albumin binds FcRn to form a tri-molecular complex with IgG. Both albumin and IgG bind noncooperatively to distinct sites on FcRn. Binding of human FcRn to Sepharose-HSA and Sepharose-hIgG was pH dependent, being maximal at pH 5.0 and nil at pH 7.0 through pH 8. The observation that FcRn binds albumin in the same pH dependent fashion as it binds IgG suggests that the mechanism by which albumin interacts with FcRn and thus is protected from degradation is identical to that of IgG, and mediated via a similarly pH-sensitive interaction with FcRn. Using SPR to measure the capacity of individual HSA domains to bind immobilized soluble hFcRn, Chaudhury showed that FcRn and albumin interact via the D-III domain of albumin in a pH-dependent manner, on a site distinct from the IgG binding site (Chaudhury, PhD dissertation, see http://www.andersonlab.com/biosketchCC.htm; Chaudhury et al. Biochemistry, ASAP Article 10.1021/bi052628y S0006-2960(05)02628-0 (Web release date: Mar. 22, 2006)).

A major disadvantage of albumin binders known in the art is their limited half-life in vivo in primates. In mice, the natural half-life of serum albumin is approximately 2 days, and different serum albumin binders have been shown to exhibit a comparable half-life, i.e. approximately 2 days. However, to the extent that known serum albumin binders have been tested in primates (i.e. of the genus Macaca, such as rhesus monkeys and cynomologus monkeys), they have exhibited a serum half-life of approximately 3 days, Reference is for example made to the data on the so-called “AlbudAb's™” (AlbudAb™ is a trademark of Domantis Ltd., Cambridge, UK) by Dr. Lucy Holt of Domantis Ltd. in the presentation “Tailoring Human Domain Antibodies for Best Practices” given on Jun. 1, 2006 during the IBC Conference “Antibodies and Beyond” on Jun. 1, 2006. In other words, the serum albumin binders for which half-life data in primates is known in the art are deficient in that they exhibit short serum half-lives in primates in vivo. These half-lives are considerably shorter than the natural half-live of serum albumin in these animals, e.g. 25% thereof.

Many therapeutics, in particular biologicals (i.e. peptide or polypeptide drugs, polynucleotides, etc.) suffer from inadequate serum half-lives in vivo. This necessitates the administration of such therapeutics at high frequencies and/or higher doses, or the use of sustained release formulations, in order to maintain the serum levels necessary for therapeutic effects. Frequent systemic administration of drugs is associated with considerable negative side effects. For example, frequent, e.g. daily, systemic injections represent a considerable discomfort to the subject, and pose a high risk of administration related infections, and may require hospitalization or frequent visits to the hospital, in particular when the therapeutic is to be administered intravenously. Moreover, in long term treatments daily intravenous injections can also lead to considerable side effects of tissue scarring and vascular pathologies caused by the repeated puncturing of vessels. Similar problems are known for all frequent systemic administrations of therapeutics, like, for example, the administration of insulin to diabetics, or interferon drugs in patients suffering from multiple sclerosis. All these factors lead to a decreased patient compliance and increased costs for the health system.

Therefore, there is a need for means to increase the serum half-life of therapeutics in primates, in particular in humans.

### SUMMARY OF THE INVENTION

The present invention solves this need by providing amino acid sequences (as well as compounds comprising the same, as defined herein), which bind to or otherwise associate with serum albumin in such a way that, when the amino acid sequence is bound to or otherwise associated with a serum albumin molecule in a primate, it exhibits a serum half-life of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of serum albumin in said primate. This significant increase in the in vivo half-life in primates makes the amino acid sequences of the invention ideal candidates to prolong the serum half-life of therapeutics attached thereto. A long serum half-life of the combined amino acid sequence and therapeutics according to the invention in turn allows for reduced frequencies of administration and/or reduced amount to be administered, bringing about significant benefits for the subject to be treated.

In one aspect, the present invention provides amino acid sequences which bind to or otherwise associate with human serum albumin in such a way that, when the amino acid sequences are bound to or otherwise associated with a human serum albumin, the amino acid sequences exhibit a serum half-life in human of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of human serum albumin. Such amino acid sequences of the invention preferably bind to human serum albumin with a dissociation constant (K_{D}) and/or with a binding affinity (K_{A}) that is as defined herein. In man, the half-life of serum albumin is about 19 days (Peters T (1996) All About Albumin. Academic Press, San Diego).

The invention also relates to compounds of the invention that comprise such an amino acid sequence and that have a half-life in human that is at least 80%, more preferably at least 90%, such as 95% or more or essentially the same as the half-life in human of the amino acid sequence present in said compound.

In one specific aspect, such amino acid sequences are preferably cross-reactive with serum albumin from at least one further species of primate, and in particular with serum albumin from at least one species of primate that is chosen from the group consisting of monkeys from the genus Macaca (such as, and in particular, cynomologus monkeys (Macaca fascicularis) and/or rhesus monkeys (Macaca mulatta)) and baboon (Papio ursinus). Preferably, such cross-reactive amino acid sequences exhibit a serum half-life in said primate of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of serum albumin in said primate. Such amino acid sequences of the invention also preferably bind to serum albumin from said primate with a dissociation constant (K_{D}) and/or with a binding affinity (K_{A}) that is as defined herein.

The invention also relates to compounds of the invention that comprise such an amino acid sequence and that have a half-life in human and/or in said at least one species of primate that is at least 80%, more preferably at least 90%, such as 95% or more or essentially the same as the half-life in human and/or said species of primate, respectively, of the amino acid sequence present in said compound.

In another aspect, the present invention provides amino acid sequences which bind to or otherwise associate with human serum albumin in such a way that, when the amino acid sequences are bound to or otherwise associated with a human serum albumin, the amino acid sequences exhibit a serum half-life in human of at least about 9 days (such as about 9 to 14 days), preferably at least about 10 days (such as about 10 to 15 days) or at least 111 days (such as about 11 to 16 days), more preferably at least about 12 days (such as about 12 to 18 days or more) or more than 14 days (such as about 14 to 19 days). Such amino acid sequences of the invention preferably bind to human serum albumin with a dissociation constant (K_{D}) and/or with a binding affinity (K_{A}) that is as defined herein.

The invention also relates to compounds of the invention that comprise such an amino acid sequence and that have a half-life in human that is at least 80%, more preferably at least 90%, such as 95% or more or essentially the same as the half-life in human of the amino acid sequence present in said compound.

In one specific aspect, such amino acid sequences are preferably cross-reactive with serum albumin from at least one further species of primate, and in particular with serum albumin from at least one species of primate that is chosen from the group consisting of monkeys from the genus Macaca (such as rhesus monkeys or cynomologus monkeys) and baboons. Preferably, such cross-reactive amino acid sequences exhibit a serum half-life in said primate of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of serum albumin in said primate. Such amino acid sequences of the invention also preferably bind to serum albumin from said primate with a dissociation constant (K_{D}) and/or with a binding affinity (K_{A}) that is as defined herein.

The invention also relates to compounds of the invention that comprise such an amino acid sequence and that have a half-life in human and/or in said at least one species of primate that is at least 80%, more preferably at least 90%, such as 95% or more or essentially the same as the half-life in human and/or said species of primate, respectively, of the amino acid sequence present in said compound.

In another aspect, the present invention relates to amino acid sequences that bind to or otherwise associate with serum albumin from at least one species of primate and that, when the half-life of serum albumin in the primate is at least about 10 days, such as between 10 and 15 days, for example about 11 to 13 days (as is for example expected for monkeys of the species Macaca, such as for cynomologus monkeys or for rhesus monkeys. For example, for rhesus monkeys, the expected half-life of serum albumin is between about 111 and 13 days, in particular about 11 to 12 days), have a serum half-life in said primate of least about 5 days (such as about 5 to 9 days), preferably at least about 6 days (such as about 6 to 10 days) or at least 7 days (such as about 7 to 11 days), more preferably at least about 8 days (such as about 8 to 12 days) or more than 9 days (such about 9 to 12 days or more). Such amino acid sequences of the invention preferably bind to serum albumin from said species of primate with a dissociation constant (K_{D}) and/or with a binding affinity (K_{A}) that is as defined herein. In one specifically preferred aspect, such amino acid sequences are cross-reactive with human serum albumin, and more preferably bind to human serum albumin with a dissociation constant (K_{D}) and/or with a binding affinity (K_{A}) that is as defined herein.

The invention also relates to compounds of the invention that comprise such an amino acid sequence and that have a half-life in said at least one species of primate that is at least 80%, more preferably at least 90%, such as 95% or more or essentially the same as the half-life in said species of primate of the amino acid sequence present in said compound.

In another aspect, the present invention relates to amino acid sequences that bind to or otherwise associate with serum albumin from at least one species of primate and that, when the half-life of serum albumin in the primate is at least about 13 days, such as between 13 and 18 days (as is for example the case for baboons, where the half-life of serum albumin is at least about 13 days, and usually about 16-18 days), have a serum half-life in said primate of least about 7 days (such as about 7 to 13 days), preferably at least about 8 days (such as about 8 to 15 days) or at least 9 days (such as about 9 to 16 days), more preferably at least about 10 days (such as about 10 to 16 days or more) or more than 13 days (such as about 13 to 18 days). Such amino acid sequences of the invention preferably bind to serum albumin from said species of primate with a dissociation constant (K_{D}) and/or with a binding affinity (K_{A}) that is as defined herein. In one specifically preferred aspect, such amino acid sequences are cross-reactive with human serum albumin, and more preferably bind to human serum albumin with a dissociation constant (K_{D}) and/or with a binding affinity (K_{A}) that is as defined herein.

The invention also relates to compounds of the invention that comprise such an amino acid sequence and that have a half-life in said at least one species of primate that is at least 80%, more preferably at least 90%, such as 95% or more or essentially the same as the half-life in said species of primate of the amino acid sequence present in said compound.

In a preferred embodiment, the invention provides amino acid sequences which:
a) bind to or otherwise associate with human serum albumin in such a way that, when the amino acid sequences are bound to or otherwise associated with a human serum albumin, the amino acid sequences exhibit a serum half-life in human of at least about 9 days (such as about 9 to 14 days), preferably at least about 10 days (such as about 10 to 15 days) or at least 111 days (such as about 11 to 16 days), more preferably at least about 12 days (such as about 12 to 18 days or more) or more than 14 days (such as about 14 to 19 days); and
b) are cross-reactive with serum albumin from at least one primate chosen from species of the genus Macaca (and in particular with serum albumin from cynomologus monkeys and/or from rhesus monkeys); and
c) have a serum half-life in said primate of at least about 5 days (such as about 5 to 9 days), preferably at least about 6 days (such as about 6 to 10 days) or at least 7 days (such as about 7 to 11 days), more preferably at least about 8 days (such as about 8 to 12 days) or more than 9 days (such about 9 to 12 days or more).

Preferably, such amino acid sequences bind to human serum albumin and/or to serum albumin from said species of primate with a dissociation constant (K_{D}) and/or with a binding affinity (K_{A}) that is as defined herein.

The invention also relates to compounds of the invention that comprise such an amino acid sequence and that have a half-life in human and/or in said at least one species of primate that is at least 80%, more preferably at least 90%, such as 95% or more or essentially the same as the half-life in human and/or said species of primate, respectively, of the amino acid sequence present in said compound.

In another preferred embodiment, the invention provides amino acid sequences which:
a) bind to or otherwise associate with human serum albumin in such a way that, when the amino acid sequences are bound to or otherwise associated with a human serum albumin, the amino acid sequences exhibit a serum half-life in human of at least about 9 days (such as about 9 to 14 days), preferably at least about 10 days (such as about 10 to 15 days) or at least 11 days (such as about 11 to 16 days), more preferably at least about 12 days (such as about 12 to 18 days or more) or more than 14 days (such as about 14 to 19 days); and
b) are cross-reactive with serum albumin from baboons; and
c) have a serum half-life in baboons of least about 7 days (such as about 7 to 13 days), preferably at least about 8 days (such as about 8 to 15 days) or at least 9 days (such as about 9 to 16 days), more preferably at least about 10 days (such as about 10 to 16 days or more) or more than 13 days (such as about 13 to 18 days).

Preferably, such amino acid sequences bind to human serum albumin and/or to serum albumin from baboon with a dissociation constant (K_{D}) and/or with a binding affinity (K_{A}) that is as defined herein.

The invention also relates to compounds of the invention that comprise such an amino acid sequence and that have a half-life in human and/or in said at least one species of primate that is at least 80%, more preferably at least 90%, such as 95% or more or essentially the same as the half-life in human and/or said species of primate, respectively, of the amino acid sequence present in said compound.

Preferably, also, the half-life of the compounds, constructs, fusion proteins, etc. comprising at least one amino acid sequence of the invention is preferably at least 80%, more preferably at least 90%, such as 95% or more or essentially the same as the half-life of the amino acid sequence of the invention present therein (i.e. in the same primate).

In a particular embodiment of the invention, the amino acid sequence of the invention (or compound comprising the same) can bind to or otherwise associate with serum albumin in such a way that, when the amino acid sequence or polypeptide construct is bound to or otherwise associated with a serum albumin molecule, the binding of said serum albumin molecule to FcRn is not (significantly) reduced or inhibited.

In a further embodiment, the amino acid sequence of the invention (or compound comprising the same) can bind to or otherwise associate with serum albumin in such a way that, when the amino acid sequence or polypeptide construct is bound to or otherwise associated with a serum albumin molecule, the half-life of the serum albumin molecule is not (significantly) reduced.

In a further embodiment the amino acid sequence of the invention (or compound comprising the same) is capable of binding to amino acid residues on serum albumin that are not involved in binding of serum albumin to FcRn, more particularly, capable of binding to amino acid residues on serum albumin that do not form part of domain III of serum albumin.

In one embodiment of the invention, the amino acid sequence is an immunoglobulin sequence or a fragment thereof, more specifically an immunoglobulin variable domain sequence or a fragment thereof, e.g. a VH-, VL- or VHH-sequence or a fragment thereof. The amino acid sequence of the invention may be a domain antibody, “dAb”, single domain antibody or Nanobody, or a fragment of any one thereof. The amino acid sequence of the invention may be a fully human, humanized, camelid, camelized human or humanized camelid sequence, and more specifically, may comprise 4 framework regions (FR1 to FR4 respectively) and 3 complementarity determining regions (CDR1 to CDR3 respectively), in which:

a) CDR1 is an amino acid sequence chosen from the group consisting of the CDR1 sequences of SEQ ID NOS: 8 to 14 and/or from the group consisting of amino acid sequences that have 2 or only 1 “amino acid difference(s)” (as defined herein) with one of the CDR1 sequences of SEQ ID NOS 8 to 14;

and/or in which:

b) CDR2 is an amino acid sequence chosen from the group consisting of the CDR2 sequences of SEQ ID NOS: 22 to 29; or from the group consisting of amino acid sequences that have at least 80%, preferably at least 90%, more preferably at least 95%, even more preferably at least 99% sequence identity (as defined herein) with one of the CDR2 sequences of SEQ ID NOS: 22 to 29; and/or from the group consisting of amino acid sequences that have 2 or only 1 “amino acid difference(s)” (as defined herein) with one of the CDR2 sequences of SEQ ID NOS 22 to 29;

and/or in which:

c1) CDR3 is an amino acid sequence chosen from the group consisting of the CDR3 sequence of SEQ ID NO: 42; the amino acid sequences that have at least 80%, preferably at least 90%, more preferably at least 95%, even more preferably at least 99% sequence identity (as defined herein) with the CDR3 sequence of SEQ ID NO: 42; and the amino acid sequences that have 3, 2 or only 1 “amino acid difference(s)” with the CDR3 sequence of SEQ ID NO:42;

or alternatively in which:

c2) CDR3 is an amino acid sequence chosen from the group consisting of the CDR3 sequences of SEQ ID NOS: 36 to 41 and/or from the group consisting of amino acid sequences that have 2 or only 1 “amino acid difference(s)” (as defined herein) with one of the CDR1 sequences of SEQ ID NOS: 36 to 41.

More specifically, the amino acid sequence according to the invention is a (single) domain antibody or a Nanobody.

The invention also relates to an amino acid sequence which has at least 80%, preferably at least 90%, more preferably at least 95%, even more preferably at least 99% sequence identity (as defined herein) with at least one of the amino acid sequences of SEQ ID NO's 50 to 64, more specifically an amino acid sequence chosen from the group consisting of PMP6A6 (ALB 1; SEQ ID NO: 52) and humanized variants thereof, including but not limited to the clones ALB 3 (SEQ ID NO: 57); ALB 4 (SEQ ID NO: 58); ALB 5 (SEQ ID NO: 59); ALB 6 (SEQ ID NO: 60); ALB 7 (SEQ ID NO: 61); ALB 8 (SEQ ID NO: 62); ALB 9 (SEQ ID NO: 63); and ALB 10 (SEQ ID NO: 64), most particularly ALB 8 (SEQ ID NO: 62).

In one embodiment, the invention relates to a compound comprising at least one amino acid sequence of the invention (also referred to herein as a “compound of the invention”), which compound may optionally further comprise at least one therapeutic moiety, comprising therapeutic moieties selected from at least one of the group consisting of small molecules, polynucleotides, polypeptides or peptides. The compound of the invention is suitable for administration to a primate with a frequency corresponding to not less than 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of serum albumin in said primate, or, alternatively, at intervals of at least 4 days (such as about 4 to 12 days or more), preferably at least 7 days (such as about 7 to 15 days or more), more preferably at least 9 days (such as about 9 to 17 days or more), such as at least 15 days (such as about 15 to 19 days or more, in particular for administration to man) or at least 17 days (such as about 17 to 19 days or more, in particular for administration to man); where such administrations are in particular made to maintain the desired level of the compound in the serum of the subject that is treated with the compound (such inter alia dependent on the compound used and/or the disease to be treated, as will be clear to the skilled person. The clinician or physician will be able to select the desired serum level and to select the dose(s) and/or amount(s) to be administered to the subject to be treated in order to achieve and/or to maintain the desired serum level in said subject, when the compound of the invention is administered at the frequencies mentioned herein. For example, such a dose can range between 1 times and 10 times the desired serum level, such as between 2 times and 4 times the desired serum level (in which the desired serum level is recalculated in a manner known per se so as to provide a corresponding dose to be administered).

The compounds of the invention may also be formulated as unit doses that are intended and/or packaged (e.g. with suitable instructions for use) for administration at the aforementioned frequencies, and such unit doses and packaged products form further aspects of the invention. Another aspect of the invention relates to the use of a compound of the invention in providing such a unit dose or packaged product (i.e. by suitably formulating and/or packaging said compound).

In a particular embodiment, the compound of the invention is a fusion protein or construct. In said fusion protein or construct the amino acid sequence of the invention may be either directly linked to the at least one therapeutic moiety or is linked to the at least one therapeutic moiety via a linker or spacer. A particular embodiment relates to a therapeutic moiety comprising an immunoglobulin sequence or a fragment thereof, more specifically a (single) domain antibody or a Nanobody.

The invention also relates to multivalent and multispecific Nanobody constructs, comprising at least one amino acid sequence of the invention which is a Nanobody and at least one further Nanobody. The Nanobody is either directly linked to the at least one further Nanobody or is linked to the at least one further Nanobody via a linker or spacer, preferably linked to the at least one further Nanobody via an amino acid sequence linker or spacer.

Furthermore, the invention relates to nucleotide sequence or nucleic acid that encode an amino acid sequence according to the invention, or the amino acid sequence of a compound according to the invention, or the multivalent and multispecific Nanobody of the invention. The invention also provides hosts or host cells that contain a nucleotide sequence or nucleic acid of the invention and/or that express (or are capable of expressing) an amino acid sequence of the invention, or the amino acid sequence of a compound according to the invention, or the multivalent and multispecific Nanobody of the invention.

Moreover, the invention relates to method for preparing an amino acid sequence, compound, or multivalent and multispecific Nanobody of the invention comprising cultivating or maintaining a host cell of the invention under conditions such that said host cell produces or expresses the said product, and optionally further comprises the said product so produced.

In one embodiment, the invention relates to a pharmaceutical composition comprising one or more selected from the group consisting of the amino acid sequence, compound, or multivalent and multispecific Nanobody of the invention, wherein said pharmaceutical composition is suitable for administration to a primate at intervals of at least about 50% of the natural half-life of serum albumin in said primate. The pharmaceutical composition may further comprise at least one pharmaceutically acceptable carrier, diluent or excipient.

The invention also encompasses medical uses and methods of treatment encompassing the amino acid sequence, compound or multivalent and multispecific Nanobody of the invention, wherein said medical use or method is characterized in that said medicament is suitable for administration at intervals of at least about 50% of the natural half-life of serum albumin in said primate, and the method comprises administration at a frequency of at least about 50% of the natural half-life of serum albumin in said primate.

The invention also relates to methods for extending or increasing the serum half-life of a therapeutic. The methods include contacting the therapeutic with any of the foregoing amino acid sequences, compounds, fusion proteins or constructs of the invention (including multivalent and multispecific Nanobodies), such that the therapeutic is bound to or otherwise associated with the amino acid sequences, compounds, fusion proteins or constructs of the invention. In some embodiments, the therapeutic is a biological therapeutic, preferably a peptide or polypeptide, in which case the step of contacting the therapeutic can include preparing a fusion protein by linking the peptide or polypeptide with the amino acid sequence, compound, fusion proteins or constructs of the invention.

These methods can further include administering the therapeutic to a primate after the therapeutic is bound to or otherwise associated with the amino acid sequence, compound, fusion protein or construct of the invention. In such methods, the serum half-life of the therapeutic in the primate is at least 1.5 times the half-life of therapeutic per se, or is increased by at least 1 hour compared to the half-life of therapeutic per se. In some preferred embodiments, the serum half-life of the therapeutic in the primate is at least 2 times, at least 5 times, at least 10 times or more than 20 times greater than the half-life of the corresponding therapeutic moiety per se. In other preferred embodiments, the serum half-life of the therapeutic in the primate is increased by more than 2 hours, more than 6 hours or more than 12 hours compared to the half-life of the corresponding therapeutic moiety per se.

Preferably, the serum half-life of the therapeutic in the primate is increased so that the therapeutic has a half-life that is as defined herein for the compounds of the invention (i.e. in human and/or in at least one species of primate).

In another aspect, the invention relates to a method for modifying a therapeutic such that the desired therapeutic level of said therapeutic is, upon suitable administration of said therapeutic so as to achieve said desired therapeutic level, maintained for a prolonged period of time.

The methods include contacting the therapeutic with any of the foregoing amino acid sequences, compounds, fusion proteins or constructs of the invention (including multivalent and multispecific Nanobodies), such that the therapeutic is bound to or otherwise associated with the amino acid sequences, compounds, fusion proteins or constructs of the invention. In some embodiments, the therapeutic is a biological therapeutic, preferably a peptide or polypeptide, in which case the step of contacting the therapeutic can include preparing a fusion protein by linking the peptide or polypeptide with the amino acid sequence, compound, fusion proteins or constructs of the invention.

These methods can further include administering the therapeutic to a primate after the therapeutic is bound to or otherwise associated with the amino acid sequence, compound, fusion protein or construct of the invention, such that the desired therapeutic level is achieved upon such administration. In such methods, the time that the desired therapeutic level of said therapeutic is maintained upon such administration is at least 1.5 times the half-life of therapeutic per se, or is increased by at least 1 hour compared to the half-life of therapeutic per se. In some preferred embodiments, the time that the desired therapeutic level of said therapeutic is maintained upon such administration is at least 2 times, at least 5 times, at least 10 times or more than 20 times greater than the half-life of the corresponding therapeutic moiety per se. In other preferred embodiments, the time that the desired therapeutic level of said therapeutic is maintained upon such administration is increased by more than 2 hours, more than 6 hours or more than 12 hours compared to the half-life of the corresponding therapeutic moiety per se.

Preferably, the time that the desired therapeutic level of said therapeutic is maintained upon such administration is increased such that the therapeutic can be administered at a frequency that is as defined herein for the compounds of the invention.

In another aspect, the invention relates to the use of a compound of the invention (as defined herein) for the production of a medicament that increases and/or extends the level of the therapeutic agent in said compound or construct in the serum of a patient such that said therapeutic agent in said compound or construct is capable of being administered at a lower dose as compared to the therapeutic agent alone (i.e. at essentially the same frequency of administration).

### BRIEF DESCRIPTION OF THE DRAWINGS

FIG. 1 is a graph of the concentration in plasma of three rhesus monkeys of the Nanobody construct (in microgram per millilitre) versus the time (in days), showing the pharmacokinetics of the Nanobody construct after administration of 2 mg/kg construct in rhesus monkeys at day 0, 1, 2, 4, 8 and 11.

FIG. 2 is a graph of the concentration in plasma of two baboons of the Nanobody construct (in microgram per millilitre) versus the time (in days), showing the pharmacokinetics of the Nanobody construct after administration of 2 mg/kg construct in baboons at day 0, 1, 2, 4, 8, 11 and 14.

### DETAILED DESCRIPTION OF THE INVENTION

In one aspect, the invention achieves this objective by providing amino acid sequences, and in particular immunoglobulin sequences, and more in particular immunoglobulin variable domain sequences, that can bind to or otherwise associate with serum albumin in such a way that, when the amino acid sequence or polypeptide construct is bound to or otherwise associated with a serum albumin molecule in a primate, it exhibits a serum half-life of at least about 50% of the natural half-life of serum albumin in said primate, preferably at least about 60%, preferably at least about 70%, more preferably at least about 80% and most preferably at least about 90%.

The serum half-life of the amino acid sequence of the invention after administration to a primate may be at least 50%, 55%, 60%, 65%, 70%, 75%, 80%, 85%, 90%, 95% or at least 100% of the natural half-life of serum albumin in said primate.

By “natural serum half-life of serum albumin in said primate” is meant the serum half-life as defined below, which serum albumin has in healthy individuals under physiological conditions. For example, the natural serum half-life of serum albumin in humans is 19 days. Smaller primates are known to have shorter natural half-lives of serum albumin, e.g. in the range of 8 to 19 days. Specific half-lives of serum albumin may be at least 4, 5, 6, 7, 8, 9, 10, 11, 12, 13, 14, 15, 16, 17, 18, or 19 days or more.

From this it follows, that for example in a human individual, an amino acid sequence of the invention shows a serum half-life in association with serum albumin of at least about 50% of 19 days, i.e. 7.6 days. In smaller primates, the serum half-life may be shorter in days, depending on the natural half-lives of serum albumin in these species.

In the present description, the term “primate” refers to both species of monkeys an apes, and includes species of monkeys such as monkeys from the genus Macaca (such as, and in particular, cynomologus monkeys (Macaca fascicularis) and/or rhesus monkeys (Macaca mulatta)) and baboon (Papio ursinus)), as well as marmosets (species from the genus Callithrix), squirrel monkeys (species from the genus Saimiri) and tamarins (species from the genus Saguinus), as well as species of apes such as chimpanzees (Pan troglodytes), and also includes man. Humans are the preferred primate according to the invention. Thus, for example, and as can be seen from the Examples below, the half-life of a Nanobody construct containing ALB-8 (SEQ ID NO: 62, an amino acid sequence of the invention) in rhesus monkeys is approximately 10 days, which is about 90% of the expected natural serum half-life of serum albumin in this species (approximately 11 days).

The half-life of an amino acid sequence or compound can generally be defined as the time taken for the serum concentration of the polypeptide to be reduced by 50%, in vivo, for example due to degradation of the sequence or compound and/or clearance or sequestration of the sequence or compound by natural mechanisms. The half-life of the amino acid sequences of the invention (and of compounds comprising the same) in the relevant species of primate can be determined in any manner known per se, such as by pharmacokinetic analysis. Suitable techniques will be clear to the person skilled in the art, and may for example generally involve the steps of suitably administering to the primate a suitable dose of the amino acid sequence or compound to be treated; collecting blood samples or other samples from said primate at regular intervals; determining the level or concentration of the amino acid sequence or compound of the invention in said blood sample; and calculating, from (a plot of) the data thus obtained, the time until the level or concentration of the amino acid sequence or compound of the invention has been reduced by 50% compared to the initial level upon dosing. Reference is for example made to the Examples below, as well as to the standard handbooks, such as Kenneth, A et al: Chemical Stability of Pharmaceuticals: A Handbook for Pharmacists and in Peters et al, Pharmacokinete analysis: A Practical Approach (1996). Reference is also made to “Pharmacokinetics”, M Gibaldi & D Perron, published by Marcel Dekker, 2nd Rev. edition (1982).

As described on pages 6 and 7 of WO 04/003019 and in the further references cited therein, the half-life can be expressed using parameters such as the t1/2-alpha, t1/2-beta and the area under the curve (AUC). In the present specification, an “increase in half-life” refers to an increase in any one of these parameters, such as any two of these parameters, or essentially all three these parameters. An “increase in half-life” in particular refers to an increase in the t1/2-beta, either with or without an increase in the t1/2-alpha and/or the AUC or both.

In another aspect, the invention provides amino acid sequences, and in particular immunoglobulin sequences, and more in particular immunoglobulin variable domain sequences, that are directed against serum albumin, preferably human serum albumin, and that have a half-life in rhesus monkeys of at least about 4, preferably at least about 7, more preferably at least about 9 days.

In a further aspect, the invention provides amino acid sequences, and in particular immunoglobulin sequences, and more in particular immunoglobulin variable domain sequences, that are directed against serum albumin, preferably human serum albumin.

In yet another aspect, the invention provides amino acid sequences, and in particular immunoglobulin sequences, and more in particular immunoglobulin variable domain sequences, that are directed against serum albumin, preferably human serum albumin, and that have a half-life in human of at least about 7, preferably at least about 15, more preferably at least about 17 days. The invention also relates to compounds of the invention that have a half-life in human that is at least 80%, more preferably at least 90%, such as 95% or more or essentially the same as the half-life of the amino acid sequence of the invention present in said compound. More in particular, the invention also relates to compounds of the invention that have a half-life in human of at least about 7, preferably at least about 15, more preferably at least about 17 days.

The invention also provides compounds comprising the amino acid sequence of the invention, in particular compounds comprising at least one therapeutic moiety in addition to the amino acid sequence of the invention. The compounds according to the invention are characterized by exhibiting a comparable serum half-life in primates to the amino acid sequence of the invention, more preferable a half-life which is at least the serum half-life of the amino acid sequence of the invention, and more preferably a half-life which is higher than the half-life of the amino acid sequence of the invention in primates.

In one aspect, the invention achieves this objective by providing amino acid sequences, and in particular immunoglobulin sequences, and more in particular immunoglobulin variable domain sequences, that can bind to or otherwise associate with serum albumin in such a way that, when the amino acid sequence or polypeptide construct is bound to or otherwise associated with a serum albumin molecule, the binding of said serum albumin molecule to FcRn is not (significantly) reduced or inhibited (i.e. compared to the binding of said serum albumin molecule to FcRn when the amino acid sequence or polypeptide construct is not bound thereto). In this aspect of the invention, by “not significantly reduced or inhibited” is meant that the binding affinity for serum albumin to FcRn (as measured using a suitable assay, such as SPR) is not reduced by more than 50%, preferably not reduced by more than 30%, even more preferably not reduced by more than 10%, such as not reduced by more than 5%, or essentially not reduced at all. In this aspect of the invention, “not significantly reduced or inhibited” may also mean (or additionally mean) that the half-life of the serum albumin molecule is not significantly reduced (as defined below).

When in this description, reference is made to binding, such binding is preferably specific binding, as normally understood by the skilled person.

When an amino acid sequence as described herein is a monovalent immunoglobulin sequence (for example, a monovalent Nanobody), said monovalent immunoglobulin sequence preferably binds to human serum albumin with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity (K_{A}) of at least 10⁷ M⁻¹, preferably at least 10⁸ M⁻¹, more preferably at least 10⁹ M⁻¹, such as at least 10¹² M⁻¹. Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

In another aspect, the invention provides amino acid sequences, and in particular immunoglobulin sequences, and more in particular immunoglobulin variable domain sequences, that can bind to or otherwise associate with serum albumin in such a way that, when the amino acid sequence or polypeptide construct is bound to or otherwise associated with a serum albumin molecule, the half-life of the serum albumin molecule is not (significantly) reduced (i.e. compared to the half-life of the serum albumin molecule when the amino acid sequence or polypeptide construct is not bound thereto). In this aspect of the invention, by “not significantly reduced” is meant that the half-life of the serum albumin molecule (as measured using a suitable technique known per se) is not reduced by more than 50%, preferably not reduced by more than 30%, even more preferably not reduced by more than 10%, such as not reduced by more than 5%, or essentially not reduced at all.

In another aspect, the invention provides amino acid sequences, and in particular immunoglobulin sequences, and more in particular immunoglobulin variable domain sequences, that are capable of binding to amino acid residues on serum albumin that are not involved in binding of serum albumin to FcRn. More in particular, this aspect of the invention provides amino acid sequences that are capable of binding to amino acid sequences of serum albumin that do not form part of domain III of serum albumin. For example, but without being limited thereto, this aspect of the invention provides amino acid sequences that are capable of binding to amino acid sequences of serum albumin that form part of domain I and/or domain II.

The amino acid sequences of the invention are preferably (single) domain antibodies or suitable for use as (single) domain antibodies, and as such may be heavy chain variable domain sequence (VH sequence) or a light chain variable domain sequence (VL sequence), and preferably are VH sequences. The amino acid sequences may for example be so-called “dAb's”.

However, according to a particularly preferred embodiment, the amino acid sequences of the present invention are Nanobodies. For a further description and definition of Nanobodies, as well as of some of the further terms used in the present description (such as, for example and without limitation, the term “directed against”) reference is made to the copending patent applications by Ablynx N. V. (such as the copending International application by Ablynx N. V. entitled “Improved Nanobodies™ against Tumor Necrosis Factor-alpha”, which has the same priority and the same international filing date as the present application); as well as the further prior art cited therein.

As such, they may be Nanobodies belonging to the “KERE”-class, to the “GLEW”-class or to the “103-P,R,S”-class (again as defined in the copending patent applications by Ablynx N. V.).

Preferably, the amino acid sequences of the present invention are humanized Nanobodies (again as defined in the copending patent applications by Ablynx N. V.).

The amino acid sequences disclosed herein can be used with advantage as a fusion partner in order to increase the half-life of therapeutic moieties such as proteins, compounds (including, without limitation, small molecules) or other therapeutic entities.

Thus, in another aspect, the invention provides proteins or polypeptides that comprise or essentially consist of an amino acid sequence as disclosed herein. In particular, the invention provides protein or polypeptide constructs that comprise or essentially consist of at least one amino acid sequence of the invention that is linked to at least one therapeutic moiety, optionally via one or more suitable linkers or spacers. Such protein or polypeptide constructs may for example (without limitation) be a fusion protein, as further described herein.

The invention further relates to therapeutic uses of protein or polypeptide constructs or fusion proteins and constructs and to pharmaceutical compositions comprising such protein or polypeptide constructs or fusion proteins.

In some embodiments the at least one therapeutic moiety comprises or essentially consists of a therapeutic protein, polypeptide, compound, factor or other entity. In a preferred embodiment the therapeutic moiety is directed against a desired antigen or target, is capable of binding to a desired antigen (and in particular capable of specifically binding to a desired antigen), and/or is capable of interacting with a desired target. In another embodiment, the at least one therapeutic moiety comprises or essentially consists of a therapeutic protein or polypeptide. In a further embodiment, the at least one therapeutic moiety comprises or essentially consists of an immunoglobulin or immunoglobulin sequence (including but not limited to a fragment of an immunoglobulin), such as an antibody or an antibody fragment (including but not limited to an ScFv fragment). In yet another embodiment, the at least one therapeutic moiety comprises or essentially consists of an antibody variable domain, such as a heavy chain variable domain or a light chain variable domain.

In a preferred embodiment, the at least one therapeutic moiety comprises or essentially consists of at least one domain antibody or single domain antibody, “dAb” or Nanobody®. According to this embodiment, the amino acid sequence of the invention is preferably also a domain antibody or single domain antibody, “dAb” or Nanobody, so that the resulting construct or fusion protein is a multivalent construct (as described herein) and preferably a multispecific construct (also as defined herein) comprising at least two domain antibodies, single domain antibodies, “dAbs” or Nanobodies® (or a combination thereof), at least one of which is directed against (as defined herein) serum albumin.

In a specific embodiment, the at least one therapeutic moiety comprises or essentially consists of at least one monovalent Nanobody® or a bivalent, multivalent, bispecific or multispecific Nanobody® construct. According to this embodiment, the amino acid sequence of the invention is preferably also a Nanobody, so that the resulting construct or fusion protein is a multivalent Nanobody construct (as described herein) and preferably a multispecific Nanobody construct (also as defined herein) comprising at least two Nanobodies, at least one of which is directed against (as defined herein) serum albumin.

According to one embodiment of the invention, the Nanobody against human serum albumin is a humanized Nanobody.

Also, when the amino acid sequences, proteins, polypeptides or constructs of the invention are intended for pharmaceutical or diagnostic use, the aforementioned are preferably directed against human serum albumin. According to one preferred, but non-limiting embodiment, the amino acid sequences, proteins, polypeptides or constructs show an affinity for human serum albumin that is higher than the affinity for mouse serum albumin (determined as described in the Examples).

According to one preferred, but non-limiting embodiment, the amino acid sequence of the invention is directed to the same epitope on human serum albumin as clone PMP6A6 (ALB-1).

According to a specific, but non-limiting embodiment, the amino acid sequence of the invention is an immunoglobulin sequence (and preferably a Nanobody) that is capable of binding to human serum albumin that consists of 4 framework regions (FR1 to FR4 respectively) and 3 complementarity determining regions (CDR1 to CDR3 respectively), in which:
a) CDR1 is an amino acid sequence chosen from the group consisting of the CDR1 sequences of SEQ ID NOS: 8 to 14 and/or from the group consisting of amino acid sequences that have 2 or only 1 “amino acid difference(s)” (as defined herein) with one of the CDR1 sequences of SEQ ID NOS 8 to 14;
   and/or in which:
   b) CDR2 is an amino acid sequence chosen from the group consisting of the CDR2 sequences of SEQ ID NOS: 22 to 29; or from the group consisting of amino acid sequences that have at least 80%, preferably at least 90%, more preferably at least 95%, even more preferably at least 99% sequence identity (as defined herein) with one of the CDR2 sequences of SEQ ID NOS: 22 to 29; and/or from the group consisting of amino acid sequences that have 2 or only 1 “amino acid difference(s)” (as defined herein) with one of the CDR2 sequences of SEQ ID NOS 22 to 29;
      and/or in which:
c1) CDR3 is an amino acid sequence chosen from the group consisting of the CDR3 sequence of SEQ ID NO: 42; the amino acid sequences that have at least 80%, preferably at least 90%, more preferably at least 95%, even more preferably at least 99% sequence identity (as defined herein) with the CDR3 sequence of SEQ ID NO: 42; and the amino acid sequences that have 3, 2 or only 1 “amino acid difference(s)” with the CDR3 sequence of SEQ ID NO:42;
   or alternatively in which:
c2) CDR3 is an amino acid sequence chosen from the group consisting of the CDR3 sequences of SEQ ID NOS: 36 to 41 and/or from the group consisting of amino acid sequences that have 2 or only 1 “amino acid difference(s)” (as defined herein) with one of the CDR1 sequences of SEQ ID NOS: 36 to 41;
   and in which the framework sequences may be any suitable framework sequences, such as the framework sequences of a (single) domain antibody and in particular of a Nanobody.

In the above amino acid sequences:

(1) any amino acid substitution is preferably a conservative amino acid substitution (as defined herein); and/or
(2) said amino acid sequence preferably only contains amino acid substitutions, and no amino acid deletions or insertions, compared to the above amino acid sequences.

Some preferred combinations of CDR sequences in the Nanobodies of the invention, and some preferred combinations of CDR and framework sequences in the Nanobodies of the invention, can be seen from Table I below.

TABLE I

preferred combinations of CDR sequences, and
preferred combination of CDR sequence and
framework sequences.

| | |
|---|---|
| CLONE | ID |

| | | |
|---|---|---|
| | | FR1 |
| PMP6A8(ALB2) | 1 | AVQLVESGGGLVQGGGSLRLACAASERIFD |
| PMP6B4 | 2 | EVQLVESGGGLVQEGGSLRLACAASERIWD |
| PMP6A6(ALB1) | 3 | AVQLVESGGGLVQPGNSLRLSCAASGFTFR |
| PMP6C1 | 4 | AVQLVDSGGGLVQPGGSLRLSCAASGFSFG |
| PMP6G8 | 5 | AVQLVESGGGLVQPGGSLRLTCTASGFTFR |
| PMP6A5 | 6 | QVQLAESGGGLVQPGGSLRLTCTASGFTFG |
| PMP6G7 | 7 | QVQLVESGGGLVQPGGSLRLSCAASGFTFS |
| | | CDR1 |
| PMP6A8(ALB2) | 8 | LNLMG |
| PMP6B4 | 9 | INLLG |
| PMP6A6(ALB1) | 10 | SFGMS |
| PMP6C1 | 11 | SFGMS |
| PMP6G8 | 12 | SFGMS |
| PMP6A5 | 13 | SFGMS |
| PMP6G7 | 14 | NYWMY |
| | | FR2 |
| PMP6A8(ALB2) | 15 | WYRQGPGNERELVA |
| PMP6B4 | 16 | WYRQGPGNERELVA |
| PMP6A6(ALB1) | 17 | WVRQAPGKEPEWVS |
| PMP6C1 | 18 | WVRQYPGKEPEWVS |
| PMP6G8 | 19 | WVRQAPGKDQEWVS |
| PMP6A5 | 20 | WVRQAPGEGLEWVS |
| PMP6G7 | 21 | WVRVAPGKGLERIS |
| | | CDR2 |
| PMP6A8(ALB2) | 22 | TCITVGDSTNYADSVKG |
| PMP6B4 | 23 | TITVGDSTSYADSVKG |
| PMP6A6(ALB1) | 24 | SISGSGSDTLYADSVKG |
| PMP6C1 | 25 | SINGRGDDTRYADSVKG |
| PMP6G8 | 26 | AISADSSTKNYADSVKG |
| PMP6A5 | 27 | AISADSSDKRYADSVKG |
| PMP6G7 | 28 | RDISTGGGYSYYADSVKG |
| | | FR3 |
| PMP6A8(ALB2) | 29 | RFTISMDYTKQTVYLHMNSLRPEDTGLYYCKI |
| PMP6B4 | 30 | RFTISRDYDKNTLYLQMNSLRPEDTGLYYCKI |
| PMP6A6(ALB1) | 31 | RFTISRDNAKTTLYLQMNSLKPEDTAVYYCTI |
| PMP6C1 | 32 | RFSISRDNAKNTLYLQMNSLKPEDTAEYYCTI |
| PMP6G8 | 33 | RFTISRDNAKKMLYLEMNSLKPEDTAVYYCVI |
| PMP6A5 | 34 | RFTISRDNAKKMLYLEMNSLKSEDTAVYYCVI |
| PMP6G7 | 35 | RFTISRDNAKNTLYLQMNSLKPEDTALYYCAK |
| | | CDR3 |
| PMP6A8(ALB2) | 36 | RRTWHSEL |
| PMP6B4 | 37 | RRTWHSEL |
| PMP6A6(ALB1) | 38 | GGSLSR |
| PMP6C1 | 39 | GRSVSRS |
| PMP6G8 | 40 | GRGSP |
| PMP6A5 | 41 | GRGSP |
| PMP6G7 | 42 | DREAQVDTLDFDY |
| | | FR4 |
| PMP6A8(ALB2) | 43 | WGQGTQVTVSS |
| PMP6B4 | 44 | WGQGTQVTVSS |
| PMP6A6(ALB1) | 45 | SSQGTQVTVSS |
| PMP6C1 | 46 | RTQGTQVTVSS |
| PMP6G8 | 47 | SSPGTQVTVSS |
| PMP6A5 | 48 | ASQGTQVTVSS |
| PMP6G7 | 49 | RGQGTQVTVSS |

Table II below lists some preferred Nanobodies of the invention. Table III below lists some preferred humanized Nanobodies of the invention.

TABLE II
preferred, but non-limiting Nanobodies of the invention.

| | | |
|---|---|---|
| PMP6A8 (ALB2) | 50 | AVQLVESGGGLVQGGGSLRLACAASERIFDLNLMGWYRQGPGNERE |
| | | LVATCITVG.DSTNYADSVKGRFTISMDYTKQTVYLHMNSLRPEDT |
| | | GLYYCKIRRTWHSELWGQGTQVTVSS |
| PMP6B4 | 51 | EVQLVESGGGLVQEGGSLRLACAASERIWDINLLGWYRQGPGNERE |
| | | LVATITVG.DSTSYADSVKGRFTISRDYDKNTLYLQMNSLRPEDTG |
| | | LYYCKIRRTWHSELWGQGTQVTVSS |
| PMP6A6 (ALB1) | 52 | AVQLVESGGGLVQPGNSLRLSCAASGFTFRSFGMSWVRQAPGKEPE |
| | | WVSSISGSGSDTLYADSVKGRFTISRDNAKTTLYLQMNSLKPEDTA |
| | | VYYCTIGGSLSRSSQGTQVTVSS |
| PMP6C1 | 53 | AVQLVDSGGGLVQPGGSLRLSCAASGFSFGSFGMSWVRQYPGKEPE |
| | | WVSSINGRGDDTRYADSVKGRFSISRDNAKNTLYLQMNSLKPEDTA |
| | | EYYCTIGRSVSRSRTQGTQVTVSS |
| PMP6G8 | 54 | AVQLVESGGGLVQPGGSLRLTCTASGFTFRSFGMSWVRQAPGKDQE |
| | | WVSAISADSSTKNYADSVKGRFTISRDNAKKMLYLEMNSLKPEDTA |
| | | VYYCVIGRGSPSSPGTQVTVSS |
| PMP6A5 | 55 | QVQLAESGGGLVQPGGSLRLTCTASGFTFGSFGMSWVRQAPGEGLE |
| | | WVSAISADSSDKRYADSVKGRFTISRDNAKKMLYLEMNSLKSEDTA |
| | | VYYCVIGRGSPASQGTQVTVSS |
| PMP6G7 | 56 | QVQLVESGGGLVQPGGSLRLSCAASGFTFSNYWMYWVRVAPGKGLE |
| | | RISRDISTGGGYSYYADSVKGRFTISRDNAKNTLYLQMNSLKPEDT |
| | | ALYYCAKDREAQVDTLDFDYRGQGTQVTVSS |

TABLE III
preferred, but non-limiting humanized Nanobodies of the invention.

| | | |
|---|---|---|
| ALB3 (ALB1 HUM1) | 57 | EVQLVESGGGLVQPGGSLRLSCAASGFTFRSFGMSWVRQAPGKEPE |
| | | WVSSISGSGSDTLYADSVKGRFTISRDNAKTTLYLQMNSLKPEDTA |
| | | VYYCTIGGSLSRSSQGTQVTVSS |
| ALB4 (ALB1 HUM2) | 58 | EVQLVESGGGLVQPGGSLRLSCAASGFTFSSFGMSWVRQAPGKEPE |
| | | WVSSISGSGSDTLYADSVKGRFTISRDNAKTTLYLQMNSLKPEDTA |
| | | VYYCTIGGSLSRSSQGTQVTVSS |
| ALB5 (ALB1 HUM3) | 59 | EVQLVESGGGLVQPGGSLRLSCAASGFTFRSFGMSWVRQAPGKGLE |
| | | WVSSISGSGSDTLYADSVKGRFTISRDNAKTTLYLQMNSLKPEDTA |
| | | VYYCTIGGSLSRSSQGTQVTVSS |
| ALB6 (ALB1 HUM1) | 60 | EVQLVESGGGLVQPGNSLRLSCAASGFTFRSFGMSWVRQAPGKGLE |
| | | WVSSISGSGSDTLYADSVKGRFTISRDNAKTTLYLQMNSLKPEDTA |
| | | VYYCTIGGSLSRSSQGTLVTVSS |
| ALB7 (ALB1 HUM2) | 61 | EVQLVESGGGLVQPGNSLRLSCAASGFTFRSFGMSWVRQAPGKGLE |
| | | WVSSISGSGSDTLYADSVKGRFTISRDNAKTTLYLQMNSLRPEDTA |
| | | VYYCTIGGSLSRSSQGTLVTVSS |
| ALB8 (ALB1 HUM3) | 62 | EVQLVESGGGLVQPGNSLRLSCAASGFTFSSFGMSWVRQAPGKGLE |
| | | WVSSISGSGSDTLYADSVKGRETISRDNAKTTLYLQMNSLRPEDTA |
| | | VYYCTIGGSLSRSSQGTLVTVSS |
| ALB9 (ALB1 HUM4) | 63 | EVQLVESGGGLVQPGNSLRLSCAASGFTFSSFGMSWVRQAPGKGLE |
| | | WVSSISGSGSDTLYADSVKGRETISRDNAKNTLYLQMNSLRPEDTA |
| | | VYYCTIGGSLSRSSQGTLVTVSS |
| ALB10 (ALB1 HUM5) | 64 | EVQLVESGGGLVQPGNSLRLSCAASGFTFSSFGMSWVRQAPGKGLE |
| | | WVSSISGSGSDTLYADSVKGRFTISRDNAKNTLYLQMNSLRPEDTA |
| | | VYYCTIGGSLSRSGQGTLVTVSS |

Thus, in another aspect, an amino acid sequence of the invention is a Nanobody, which has at least 80%, preferably at least 90%, more preferably at least 95%, even more preferably at least 99% sequence identity (as defined herein) with at least one of the amino acid sequences of SEQ ID NO's 50 to 64.

Thus, in another aspect, an amino acid sequence of the invention is a Nanobody, which has at least 80%, preferably at least 90%, more preferably at least 95%, even more preferably at least 99% sequence identity (as defined herein) with at least one of the amino acid sequences of SEQ ID NO's 50 to 64, in which:
the CDR1 sequences present in such Nanobodies are chosen from the CDR1 sequences of SEQ ID NOS: 8 to 14 or from amino acid sequences with only 1 amino acid difference with such a CDR1 sequence;
the CDR2 sequences present in such Nanobodies are chosen from the CDR1 sequences of SEQ ID NOS: 22 to 28 or from amino acid sequences with only 1 amino acid difference with such a CDR2 sequence;
and the CDR1 sequences present in such Nanobodies are chosen from the CDR1 sequences of SEQ ID NOS: 23 to 42 or from amino acid sequences with only 1 amino acid difference with such a CDR3 sequence.

In another aspect, an amino acid sequence of the invention is a Nanobody, which has at least 80%, preferably at least 90%, more preferably at least 95%, even more preferably at least 99% sequence identity (as defined herein) with at least one of the amino acid sequences of SEQ ID NO's 50 to 64, in which:
the CDR1 sequences present in such Nanobodies are chosen from the CDR1 sequences of SEQ ID NOS: 8 to 14;
the CDR2 sequences present in such Nanobodies are chosen from the CDR1 sequences of SEQ ID NOS: 22 to 28;
and the CDR1 sequences present in such Nanobodies are chosen from the CDR1 sequences of SEQ ID NOS: 23 to 42.

One particularly preferred group of Nanobodies for use in the present invention comprises clone PMP6A6 (ALB 1; SEQ ID NO: 52) and humanized variants thereof, including but not limited to the clones ALB 3 (SEQ ID NO: 57); ALB 4 (SEQ ID NO: 58); ALB 5 (SEQ ID NO: 59); ALB 6 (SEQ ID NO: 60); ALB 7 (SEQ ID NO: 61); ALB 8 (SEQ ID NO: 62); ALB 9 (SEQ ID NO: 63); and ALB 10 (SEQ ID NO: 64), of which ALB 8 (SEQ ID NO: 62) is particularly preferred.

Thus, in one preferred aspect, the invention relates to an amino acid sequence, which has at least 80%, preferably at least 90%, more preferably at least 95%, even more preferably at least 99% sequence identity (as defined herein) with at least one of the amino acid sequences of SEQ ID NO's 52 and 57 to 64.

In another preferred aspect, the amino acid sequence of the invention is an immunoglobulin sequence (and preferably a Nanobody) that is capable of binding to human serum albumin that consists of 4 framework regions (FR1 to FR4 respectively) and 3 complementarity determining regions (CDR1 to CDR3 respectively), in which:

a) CDR1 comprises, is or essentially consists of:
the amino acid sequence SFGMS; or
an amino acid sequence that has at least 80%, preferably at least 90%, more preferably at least 95%, even more preferably at least 99% sequence identity with the amino acid sequence SFGMS; or
an amino acid sequences that has 2 or only 1 amino acid difference(s) with the amino acid sequence SFGMS;
   and/or in which:
   b) CDR2 comprises, is or essentially consists of:
the amino acid sequence SISGSGSDTLYADSVKG; or
an amino acid sequence that has at least 80%, preferably at least 90%, more preferably at least 95%, even more preferably at least 99% sequence identity with the amino acid sequence SISGSGSDTLYADSVKG; or
an amino acid sequences that has 2 or only 1 amino acid difference(s) with the amino acid sequence SISGSGSDTLYADSVKG;
   and/or in which:
   c) CDR3 comprises, is or essentially consists of:
the amino acid sequence GGSLSR; or
an amino acid sequence that has at least 80%, preferably at least 90%, more preferably at least 95%, even more preferably at least 99% sequence identity with the amino acid sequence GGSLSR; or
an amino acid sequences that has 2 or only 1 amino acid difference(s) with the amino acid sequence GGSLSR.

In particular, the invention relates to such a Nanobody, in which:
CDR1 comprises or is the amino acid sequence SFGMS;

and/or in which
CDR2 comprises or is the amino acid sequence SISGSGSDTLYADSVKG;

and/or in which:
CDR3 comprises or is the amino acid sequence SPSGFN.

More in particular, the invention relates to such a Nanobody, in which
CDR1 comprises or is the amino acid sequence SFGMS; and CDR3 comprises or is comprises the amino acid sequence GGSLSR;

and/or in which:
CDR1 comprises or is the amino acid sequence SFGMS; and CDR2 comprises or is the amino acid sequence SISGSGSDTLYADSVKG;

and/or in which:
CDR2 comprises or is the amino acid sequence SISGSGSDTLYADSVKG; and

CDR3 comprises or is the amino acid sequence GGSLSR.

Even more in particular, the invention relates to such a Nanobody, in which CDR1 comprises or is the amino acid sequence SFGMS; CDR2 comprises or is the amino acid sequence SISGSGSDTLYADSVKG and CDR3 comprises or is the amino acid sequence GGSLSR.

These amino acid sequences again preferably have at least 80%, preferably at least 90%, more preferably at least 95%, even more preferably at least 99% sequence identity (as defined herein) with at least one of the amino acid sequences of SEQ ID NO's 52 and 57 to 64.

Also, again, these amino acid sequences are preferably humanized, as described in the co-pending applications by Ablynx N. V. Some preferred humanizing substitutions will be clear from the skilled person, for example from comparing the non-humanized sequence of SEQ ID NO: 52 with the corresponding humanized sequences of SEQ ID NOS: 57-64.

When the amino acid sequence is an immunoglobulin sequence such as a immunoglobulin variable domain sequence, a suitable (i.e. suitable for the purposes mentioned herein) fragment of such a sequence may also be used. For example, when the amino acid sequence is a Nanobody, such a fragment may essentially be as described in WO 04/041865.

The invention also relates to a protein or polypeptide that comprises or essentially consists of an amino acid sequence as described herein, or a suitable fragment thereof.

As mentioned herein, the amino acid sequences described herein can be used with advantage as a fusion partner in order to increase the half-life of therapeutic moieties such as proteins, compounds (including, without limitation, small molecules) or other therapeutic entities. Thus, one embodiment of the invention relates to a construct or fusion protein that comprises at least one amino acid sequence of the invention and at least one therapeutic moieties. Such a construct or fusion protein preferably has increased half-life, compared to the therapeutic moiety per se. Generally, such fusion proteins and constructs can be (prepared and used) as described in the prior art cited above, but with an amino acid sequence of the invention instead of the half-life increasing moieties described in the prior art.

Generally, the constructs or fusion proteins described herein preferably have a half-life that is at least 1.5 times, preferably at least 2 times, such as at least 5 times, for example at least 10 times or more than 20 times, greater than the half-life of the corresponding therapeutic moiety per se.

Also, preferably, any such fusion protein or construct has a half-life that is increased with more than 1 hour, preferably more than 2 hours, more preferably of more than 6 hours, such as of more than 12 hours, compared to the half-life of the corresponding therapeutic moiety per se.

Also, preferably, any fusion protein or construct has a half-life that is more than 1 hour, preferably more than 2 hours, more preferably of more than 6 hours, such as of more than 12 hours, and for example of about one day, two days, one week, two weeks or three weeks, and preferably no more than 2 months, although the latter may be less critical.

Also, as mentioned above, when the amino acid sequence of the invention is a Nanobody, it can be used to increase the half-life of other immunoglobulin sequences, such as domain antibodies, single domain antibodies, “dAb's” or Nanobodies.

Thus, one embodiment of the invention relates to a construct or fusion protein that comprises at least one amino acid sequence of the invention and at least one immunoglobulin sequence, such as a domain antibodies, single domain antibodies, “dAb's” or Nanobodies. The immunoglobulin sequence is preferably directed against a desired target (which is preferably a therapeutic target), and/or another immunoglobulin sequence that useful or suitable for therapeutic, prophylactic and/or diagnostic purposes.

Thus, in another aspect, the invention relates to a multispecific (and in particular bispecific) Nanobody constructs that comprises at least one Nanobody as described herein, and at least one other Nanobody, in which said at least one other Nanobody is preferably directed against a desired target (which is preferably a therapeutic target), and/or another Nanobody that useful or suitable for therapeutic, prophylactic and/or diagnostic purposes.

For a general description of multivalent and multispecific polypeptides containing one or more Nanobodies and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 96/34103 and WO 99/23221. Some other examples of some specific multispecific and/or multivalent polypeptide of the invention can be found in the co-pending applications by Ablynx N. V. In particular, for a general description of multivalent and multispecific constructs comprising at least one Nanobody against a serum protein for increasing the half-life, of nucleic acids encoding the same, of compositions comprising the same, of the preparation of the aforementioned, and of uses of the aforementioned, reference is made to the International application WO 04/041865 by Ablynx N. V. mentioned above. The amino acid sequences described herein can generally be used analogously to the half-life increasing Nanobodies described therein.

In one non-limiting embodiment, said other Nanobody is directed against tumor necrosis factor alpha (TNF-alpha), in monomeric and/or multimeric (i.e. trimeric) form. Some examples of such Nanobody constructs can be found in the copending International application by Ablynx N. V. entitled “Improved Nanobodies™ against Tumor Necrosis Factor-alpha”, which has the same priority and the same international filing date as the present application.

The invention also relates to nucleotide sequences or nucleic acids that encode amino acid sequences, compounds, fusion proteins and constructs described herein. The invention further includes genetic constructs that include the foregoing nucleotide sequences or nucleic acids and one or more elements for genetic constructs known per se. The genetic construct may be in the form of a plasmid or vector. Again, such constructs can be generally as described in the co-pending patent applications by Ablynx N. V. described herein, such as WO 04/041862 or the copending International application by Ablynx N. V. entitled “Improved Nanobodies™ against Tumor Necrosis Factor-alpha”.

The invention also relates to hosts or host cells that contain such nucleotide sequences or nucleic acids, and/or that express (or are capable of expressing), the amino acid sequences, compounds, fusion proteins and constructs described herein. Again, such host cells can be generally as described in the co-pending patent applications by Ablynx N. V. described herein, such as WO 04/041862 or the copending International application by Ablynx N. V. entitled “Improved Nanobodies™ against Tumor Necrosis Factor-alpha”.

The invention also relates to a method for preparing an amino acid sequence, compound, fusion protein or construct as described herein, which method comprises cultivating or maintaining a host cell as described herein under conditions such that said host cell produces or expresses an amino acid sequence, compound, fusion protein or construct as described herein, and optionally further comprises isolating the amino acid sequence, compound, fusion protein or construct so produced. Again, such methods can be performed as generally described in the co-pending patent applications by Ablynx N. V. described herein, such as WO 04/041862 or the copending International application by Ablynx N. V. entitled “Improved Nanobodies™ against Tumor Necrosis Factor-alpha”.

The invention also relates to a pharmaceutical composition that comprises at least one amino acid sequence, compound, fusion protein or construct as described herein, and optionally at least one pharmaceutically acceptable carrier, diluent or excipient. Such preparations, carriers, excipients and diluents may generally be as described in the co-pending patent applications by Ablynx N. V. described herein, such as WO 04/041862 or the copending International application by Ablynx N. V. entitled “Improved Nanobodies™ against Tumor Necrosis Factor-alpha”.

However, since the amino acid sequences, compounds, fusion proteins or constructs described herein have an increased half-life, they are preferably administered to the circulation. As such, they can be administered in any suitable manner that allows the amino acid sequences, compound, fusion proteins or constructs to enter the circulation, such as intravenously, via injection or infusion, or in any other suitable manner (including oral administration, administration through the skin, transmucosal administration, intranasal administration, administration via the lungs, etc) that allows the amino acid sequences, compounds, fusion proteins or constructs to enter the circulation. Suitable methods and routes of administration will be clear to the skilled person, again for example also from the teaching of WO 04/041862 or the copending International application by Ablynx N. V. entitled “Improved Nanobodies™ against Tumor Necrosis Factor-alpha.

Thus, in another aspect, the invention relates to a method for the prevention and/or treatment of at least one disease or disorder that can be prevented or treated by the use of a compound, fusion protein or construct as described herein, which method comprises administering, to a subject in need thereof, a pharmaceutically active amount of an amino acid sequence, compound, fusion protein or construct of the invention, and/or of a pharmaceutical composition comprising the same. The diseases and disorders that can be prevented or treated by the use of an amino acid sequence, compound, fusion protein or construct as described herein will generally be the same as the diseases and disorders that can be prevented or treated by the use of the therapeutic moiety that is present in the amino acid sequence, compound, fusion protein or construct of the invention.

The subject to be treated may be any primate, but is in particular a human being. As will be clear to the skilled person, the subject to be treated will in particular be a person suffering from, or at risk from, the diseases and disorders mentioned herein.

More specifically, the present invention relates to a method of treatment wherein the frequency of administering the amino acid sequence, compound, fusion protein or construct of the invention is at least 50% of the natural half-life of serum albumin in said primate, preferably at least 60%, preferably at least 70%, more preferably at least 80% and most preferably at least 90%.

Specific frequencies of administration to a primate, which are within the scope of the present invention are at least 50%, 55%, 60%, 65%, 70%, 75%, 80%, 85%, 90%, 95% or at least 100% of the natural half-life of serum albumin in said primate as defined above.

In other words, specific frequencies of administration which are within the scope of the present invention are every 4, 5, 6, 7, 8, 9, 10, 11, 12, 13, 14, 15, 16, 17, 18, or 19 days.

Without limitation, the frequencies of administration referred to above are in particular suited for maintaining a desired level of the amino acid sequence, compound, fusion protein or construct in the serum of the subject treated with the amino acid sequence, compound, fusion protein or construct, optionally after administration of one or more (initial) doses that are intended to establish said desired serum level. As will be clear to the skilled person, the desired serum level may inter alia be dependent on the amino acid sequence, compound, fusion protein or construct used and/or the disease to be treated. The clinician or physician will be able to select the desired serum level and to select the dose(s) and/or amount(s) to be administered to the subject to be treated in order to achieve and/or to maintain the desired serum level in said subject, when the amino acid sequence, compound, fusion protein or construct of the invention is administered at the frequencies mentioned herein.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any primate, but is in particular a human being. As will be clear to the skilled person, the subject to be treated will in particular be a person suffering from, or at risk from, the diseases and disorders treatable by the therapeutic moiety mentioned herein.

In another embodiment, the invention relates to a method for immunotherapy, and in particular for passive immunotherapy, which method comprises administering, to a subject suffering from or at risk of the diseases and disorders mentioned herein, a pharmaceutically active amount of an amino acid sequence, compound, fusion protein or construct of the invention, and/or of a pharmaceutical composition comprising the same.

The invention also relates to methods for extending or increasing the serum half-life of a therapeutic. In these methods, the therapeutic is contacted with any of the amino acid sequences, compounds, fusion proteins or constructs of the invention, including multivalent and multispecific Nanobodies, such that the therapeutic is bound to or otherwise associated with the amino acid sequences, compounds, fusion proteins or constructs.

The therapeutic and the amino acid sequences, compounds, fusion proteins or constructs can be bound or otherwise associated in various ways known to the skilled person. In the case of biological therapeutics, such as a peptide or polypeptide, the therapeutic can be fused to the amino acid sequences, compounds, fusion proteins or constructs according to methods known in the art. The therapeutic can be directly fused, or fused using a spacer or linker molecule or sequence. The spacer or linker are, in preferred embodiments, made of amino acids, but other non-amino acid spacers or linkers can be used as is well known in the art. Thus, the step of contacting the therapeutic can include preparing a fusion protein by linking the peptide or polypeptide with the amino acid sequences, compounds, fusion proteins or constructs of the invention, including multivalent and multispecific Nanobodies.

The therapeutic also can be bound directly by the amino acid sequences, compounds, fusion proteins or constructs of the invention. As one example, a multivalent and multispecific Nanobody can include at least one variable domain that binds serum albumin and at least one variable domain that binds the therapeutic.

The methods for extending or increasing serum half-life of a therapeutic can further include administering the therapeutic to a primate after the therapeutic is bound to or otherwise associated with the amino acid sequence, compound, fusion proteins or constructs of the invention. In such methods the half-life of the therapeutic is extended or increased by significant amounts, as is described elsewhere herein.

The amino acid sequence, compound, fusion protein or construct and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific Nanobody or polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more amino acid sequences, compounds, fusion proteins or constructs of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency and/or the half-life of the specific amino acid sequences, compounds, fusion proteins or constructs to be used, the specific route of administration and the specific pharmaceutical formulation or composition used, the Nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single Nanobody or polypeptide of the invention will be used. It is however within the scope of the invention to use two or more Nanobodies and/or polypeptides of the invention in combination.

The Nanobodies and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

In particular, the Nanobodies and polypeptides of the invention may be used in combination with other pharmaceutically active compounds or principles that are or can be used for the prevention and/or treatment of the diseases and disorders that can be prevented or treated with the fusion proteins or constructs of the invention, and as a result of which a synergistic effect may or may not be obtained.

The effectiveness of the treatment regimen used according to the invention may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

The invention will now be further described by means of the following non-limiting Examples and the attached Figures.

### EXAMPLES

### Example 1

### Identification of Serum Albumin Specific Nanobodies

The albumin specific nanobodies were identified from a llama immunized with human serum albumin. Screening of individual nanobodies was performed by ELISA using human, rhesus and mouse albumin, yielding a panel of nanobodies cross-reacting with the serum albumin of various species.

### Example 2

### Biacore Analysis

Binding of nanobodies to serum albumin was characterised by surface plasmon resonance in a Biacore 3000 instrument. Serum albumin from different species was covalently bound to CM5 sensor chips surface via amine coupling until an increase of 250 response units was reached. Remaining reactive groups were inactivated. Nanobody binding was assessed at one concentration (1 in 20 diluted). Each nanobody was injected for 4 minutes at a flow rate of 45 μl/min to allow for binding to chip-bound antigen. Binding buffer without nanobody was sent over the chip at the same flow rate to allow spontaneous dissociation of bound nanobody for 4 hours. K_{off}-values were calculated from the sensorgrams obtained for the different nanobodies. The nanobodies tested are ranked according to k_{off}-values, see Table IV below:

TABLE IV

| Class | Human | Rhesus | Mouse |
|---|---|---|---|
| C | PMP6A8 | PMP6A8 | PMP6B4 |
| C | PMP6B4 | PMP6B4 | PMP6A8 |
| B | PMP6A6 | PMP6A6 | PMP6A6 |
| B | PMP6C1 | PMP6C1 | PMP6C1 |
| A | PMP6G8 | PMP6G8 | PMP6G8 |
| A | PMP6A5 | PMP6A5 | PMP6A5 |
| D | PMP6G7 | PMP6G7 | PMP6G7 |

In a follow-up experiment, binding was assayed as described above except that series of different concentrations were used. Each concentration was injected for 4 minutes at a flow rate of 45 μl/min to allow for binding to chip-bound antigen. Binding buffer without analyte was sent over the chip at the same flow rate to allow for dissociation of bound nanobody. After 15 minutes, remaining bound analyte was removed by injection of the regeneration solution (25 mM NaOH).

From the sensorgrams obtained for the different concentrations of each analyte K_{D}-values were calculated via steady state affinity when equilibrium was reached.

Results are summarized in Table V. Cross-reactivity is observed for both ALB1 and ALB2. The highest affinity is observed for ALB2 on human and rhesus TNFα. However, the difference in affinity for human/rhesus versus mouse serum albumin is more pronounced for ALB2 (factor 400), while for ALB1 a difference of a factor 12 is observed.

TABLE V

| | | Human | Rhesus | Mouse |
|---|---|---|---|---|
| | | albumin | albumin | albumin |
| ALB1 | KD (nM) | 0.57 | 0.52 | 6.5 |
| | ka | 1.11E+06 | 1.05E+06 | 1.11E+06 |
| | (1/Ms) | | | |
| | kd (1/s) | 6.30E−04 | 5.46E−04 | 7.25E−03 |
| ALB2 | KD (nM) | 0.092 | 0.036 | 15.7 |
| | ka | 8.15E+05 | 1.94E+06 | 1.95E+05 |
| | (1/Ms) | | | |
| | kd (1/s) | 7.52E−05 | 7.12E−05 | 3.07E−03 |

### Example 3

### Half-Life in Rhesus Monkeys

The pharmacokinetic properties of a trivalent bispecific Nanobody construct comprising the humanized anti-human serum albumin Nanobody ALB-8 (SEQ ID NO: 62) were investigated in rhesus monkeys. On day 0, three monkeys received 2 mg/kg of the construct in. Plasma samples were taken from the monkeys upon administration and on days 1, 2, 4, 8, 11 and 14 following administration (as set out below) and were analyzed to determine the pharmacokinetic profile. The PK profiles in all monkeys were similar, with a calculated half-life of approximately 10 days. This calculated half-life is in the range of the presumed half-life of albumin in rhesus monkeys.

Three rhesus monkeys were acclimatized 4 weeks prior to the study for acclimatization. On day 0, the monkeys received 2 mg/kg of the construct via an intravenous infusion into the vena cephalica of the right or left arm using indwelling catheters and an infusion pump. The dose was administered as a slow bolus in a volume of 2 ml/kg over 5 minutes. During each dosing cycle blood samples were taken at the following times:

prior to infusion:
40 min before start of slow bolus

after starting infusion:
5 and 30 minutes after starting slow bolus
1, 2, 4, and 8 hours after starting slow bolus
1, 2, 4, 8, and 11 days post-dosing

2 ml whole blood were withdrawn from the vena cephalica of the left or right arm, which was not used for application, or from the vena saphena magna from the left or right hind limb in order to obtain approximately 800 μL Na-Heparin plasma from each animal at each sampling time.

For the PK analysis, a 96-well Maxisorp plate was coated with 2 μg/ml NeutrAvidin (Pierce) at 100 μl/well in PBS ON at 4° C. Plates were blocked with PBS, 1% casein using 200 μl/well for 2 h at RT. Biotinylated antigen at 0.4 μg/ml in PBS, 0.2% casein was added to the wells and incubated for 1 h at RT. Plasma samples were diluted in a non-coated plate and incubated for 15 min at RT. 100 μl of each diluted plasma sample was then transferred into the previously prepared wells, followed by incubation for 2 h at RT. Bound construct was detected using a polyclonal rabbit anti-Nanobody antibody (custom-made by Dabio, Germany by immunizing rabbits with various Nanobodies) diluted 1/2000 followed by addition of anti-rabbit IgG alkaline phosphatase antibody (diluted 1/2000, Sigma, A1902) and 2 mg/ml pNPP (paranitrophenylphosphate) as substrate. The absorbance is measured at 405 nm.

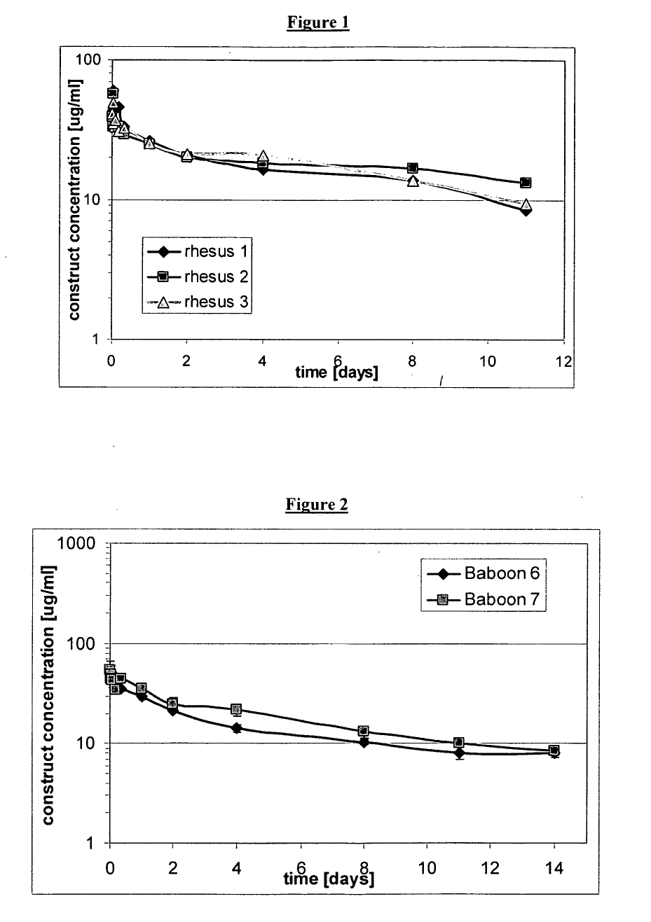

The concentration of the construct in plasma samples was determined by comparison with a standard curve of the construct diluted in an appropriate concentration of rhesus monkey plasma. The results are shown in FIG. 1. From this data, it can be seen that in general, all monkeys showed a pharmacokinetic profile with a terminal half-life of approximately 10 days, which is within the range of the presumed half-life of albumin in rhesus monkeys: the calculated terminal half-lives (t1/2 cycle I [d]) of the Nanobody construct were between 8.0 and 12.5 days.

### Example 4

### Half-Life in Baboons

The pharmacokinetic properties of the construct used in Example 3 were tested in baboons, essentially in the same manner as described in Example 3 for the rhesus monkey studies. On day 0, two baboons received 2 mg/kg of the construct. Plasma samples were taken from the baboons monkeys upon administration and on days 1, 2, 4, 8, 11 and 14 following administration (as set out below) and were analyzed to determine the pharmacokinetic behaviour of the construct. The pharmacokinetic profile of the construct in baboons was similar to the profile in rhesus monkeys, and was characterized by an average half-life of about 10 days, calculated from the PK data.

Two male juvenile baboons were used in this study. The animals weighed approximately 10-15 kg and were disease free for at least 6 weeks prior to use. To enable handling, the baboons were sedated with approximately 1 mg/kg ketamine hydrochloride. On day 0, the baboons received of 2 mg/kg of the construct via an intravenous infusion into the vena cephalica of the right or left arm using indwelling catheters and an infusion pump. The dose was administered as a slow bolus in a volume of 2 ml/kg over 5 minutes. During each of the construct dosing cycle blood samples were taken at the following times:

Prior to infusion:
40 min before start of slow bolus

After starting infusion:
5 and 30 minutes after starting slow bolus
1, 2, 4, and 8 hours after starting slow bolus
1, 2, 4, 8, and 11 days post-dosing

2 ml whole blood were withdrawn from the vena cephalica of the left or right arm, which was not used for application, or from the vena saphena magna from the left or right hind limb in order to obtain approximately 800 μL Na-Heparin plasma from each animal at each sampling time.

A 96-well Maxisorp plate was coated with 2 μg/ml NeutrAvidin (Pierce) at 100 μl/well in PBS ON at 4° C. Plates were blocked with PBS, 1% casein using 200 μl/well for 2 h at RT. Biotinylated antigen in PBS, 0.2% casein was added to the wells and incubated for 1 h at RT. Plasma samples were diluted in a non-coated plate and incubated for 15 min at RT. 100 μl of each diluted plasma sample was then transferred into the previously prepared wells, followed by incubation for 2 h at RT.

Bound construct was detected using a polyclonal rabbit anti-Nanobody antibody (as above) diluted 1/2000 followed by addition of anti-rabbit IgG alkaline phosphatase antibody (diluted 1/2000, Sigma, A1902) and 2 mg/ml pNPP as substrate. The absorbance is measured at 405 nm. The concentration of the construct in plasma samples was determined by comparison with a standard curve of the construct diluted in an appropriate concentration of monkey plasma.

FIG. 2 gives a graphic representation of the pharmacokinetics of the construct in the baboons. The calculated terminal half-life of the construct was about 11 days, which is generally comparable with the PK observed in rhesus monkeys. The ALB008 building block in the construct has an affinity of 36 nM for baboon albumin, as determined by BIAcore, resulting in an extension of the terminal half-life of the Nanobody™ from less than 1 hour to about the half-life of albumin, which is reported to be 16 to 18 days in baboons (Cohen, Biochemistry 64, 1956).

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference, in particular for the teaching that is referenced hereinabove.

1. Amino acid sequence which binds to or otherwise associates with serum albumin in such a way that, when the amino acid sequence is bound to or otherwise associated with a serum albumin molecule in a primate, said amino acid sequence exhibits a serum half-life of at least 50% of the natural serum half-life of serum albumin in said primate.

2. The amino acid sequence according to claim 1, wherein said amino acid sequence exhibits a serum half-life of at least 60% of the natural serum half-life of serum albumin in said primate.

3. The amino acid sequence according to claim 1, wherein said amino acid sequence exhibits a serum half-life of at least 80% of the natural serum half-life of serum albumin in said primate.

4. The amino acid sequence according to claim 1, wherein said amino acid sequence exhibits a serum half-life of at least 90% of the natural serum half-life of serum albumin in said primate.

5. The amino acid sequence according to claim 1, wherein said amino acid sequence exhibits a serum half-life of at least 4 days.

6. The amino acid sequence according to claim 5, wherein said amino acid sequence exhibits a serum half-life of at least 7 days.

7. The amino acid sequence according to claim 5 or 6, wherein said amino acid sequence exhibits a serum half-life of at least 9 days.

8. The amino acid sequence according to claim 1, that can bind to or otherwise associate with serum albumin in such a way that, when the amino acid sequence is bound to or otherwise associated with a serum albumin molecule, the binding of said serum albumin molecule to FcRn is not (significantly) reduced or inhibited.

9. The amino acid sequence according to claim 1 any one of claims 1 to 8, that can bind to or otherwise associate with serum albumin in such a way that, when the amino acid sequence is bound to or otherwise associated with a serum albumin molecule, the half-life of the serum albumin molecule is not (significantly) reduced.

10. The amino acid sequence according to claim 1, that is capable of binding to amino acid residues on serum albumin that are not involved in binding of serum albumin to FcRn.

11. The amino acid sequence according to claim 1, that is capable of binding to amino acid residues on serum albumin that do not form part of domain III of serum albumin.

12. The amino acid sequence according to claim 1, which is an immunoglobulin sequence or a fragment thereof.

13. The amino acid sequence according to claim 12, which is an immunoglobulin variable domain sequence or a fragment thereof.

14. The amino acid sequence according to claim 13, which is a VH-, VL- or VHH-sequence or a fragment thereof.

15. The amino acid sequence according to claim 11, wherein said immunoglobulin sequence is a domain antibody, “dAb”, single domain antibody or Nanobody, or a fragment of any one thereof.

16. The amino acid sequence according to claim 1, which is a fully human, humanized, camelid, camelized human or humanized camelid sequence.

17. The amino acid sequence according to claim 1, wherein said amino acid sequence comprises 4 framework regions (FR1 to FR4 respectively) and 3 complementarity determining regions (CDR1 to CDR3 respectively), in which:
a) CDR1 is an amino acid sequence chosen from the group consisting of the CDR1 sequences of SEQ ID NOS: 8 to 14 and/or from the group consisting of amino acid sequences that have 2 or only 1 “amino acid difference(s)” (as defined herein) with one of the CDR1 sequences of SEQ ID NOS 8 to 14;
and in which:
b) CDR2 is an amino acid sequence chosen from the group consisting of the CDR2 sequences of SEQ ID NOS: 22 to 29; or from the group consisting of amino acid sequences that have at least 80%, preferably at least 90%, more preferably at least 95%, even more preferably at least 99% sequence identity (as defined herein) with one of the CDR2 sequences of SEQ ID NOS: 22 to 29; and/or from the group consisting of amino acid sequences that have 2 or only 1 “amino acid difference(s)” (as defined herein) with one of the CDR2 sequences of SEQ ID NOS 22 to 29;
and in which:
c1) CDR3 is an amino acid sequence chosen from the group consisting of the CDR3 sequence of SEQ ID NO: 42; the amino acid sequences that have at least 80%, preferably at least 90%, more preferably at least 95%, even more preferably at least 99% sequence identity (as defined herein) with the CDR3 sequence of SEQ ID NO: 42; and the amino acid sequences that have 3, 2 or only 1 “amino acid difference(s)” with the CDR3 sequence of SEQ ID NO:42;
or alternatively in which:
c2) CDR3 is an amino acid sequence chosen from the group consisting of the CDR3 sequences of SEQ ID NOS: 36 to 41 and/or from the group consisting of amino acid sequences that have 2 or only 1 “amino acid difference(s)” (as defined herein) with one of the CDR1 sequences of SEQ ID NOS: 36 to 41.

18. The amino acid sequence according to claim 17, which is a (single) domain antibody or a Nanobody.

19. The amino acid sequence according to claim 17, which has at least 80%, preferably at least 90%, more preferably at least 95%, even more preferably at least 99% sequence identity (as defined herein) with at least one of the amino acid sequences of SEQ ID NO's 50 to 64.

20. The amino acid sequence according to claim 19, which is chosen from the group consisting of PMP6A6 (ALB 1; SEQ ID NO: 52) and humanized variants thereof, preferably ALB 3 (SEQ ID NO: 57); ALB 4 (SEQ ID NO: 58); ALB 5 (SEQ ID NO: 59); ALB 6 (SEQ ID NO: 60); ALB 7 (SEQ ID NO: 61); ALB 8 (SEQ ID NO: 62); ALB 9 (SEQ ID NO: 63); or ALB 10 (SEQ ID NO: 64).

21. The amino acid sequence according to claim 20, which is ALB 8 (SEQ ID NO: 62).

22. Compound comprising the amino acid sequence of claim 1.

23. The compound according to claim 22, wherein said compound further comprises at least one therapeutic moiety.

24. The compound according to claim 23, wherein said therapeutic moiety is selected from at least one of the group consisting of small molecules, polynucleotides, polypeptides or peptides.

25. The compound according to claim 22, which is a fusion protein or construct.

26. The compound according to claim 25, wherein in said fusion protein or construct the amino acid sequence is either directly linked to the at least one therapeutic moiety or is linked to the at least one therapeutic moiety via a linker or spacer.

27. The compound according to claim 22, in which the therapeutic moiety comprises an immunoglobulin sequence or a fragment thereof.

28. The compound according to claim 27, in which the therapeutic moiety comprises a (single) domain antibody or a Nanobody.

29. Multivalent and multispecific Nanobody construct, comprising at least one amino acid sequence according to claim 1 which is a Nanobody and at least one further Nanobody.

30. The multivalent and multispecific Nanobody construct according to claim 29, in which the amino acid sequence that is a Nanobody is either directly linked to the at least one further Nanobody or is linked to the at least one further Nanobody via a linker or spacer.

31. The multivalent and multi specific Nanobody construct according to claim 30, in which the amino acid sequence that is a Nanobody is linked to the at least one further Nanobody via a linker or spacer, and in which the linker is an amino acid sequence.

32. Nucleotide sequence or nucleic acid that encodes the amino acid sequence according to claim 1.

33. Hosts or host cells that contain a nucleotide sequence or nucleic acid according to claim 32.

34. Method for preparing an amino acid sequence which method comprises cultivating or maintaining a host cell according to claim 33 under conditions such that said host cell produces or expresses the amino acid sequence, and optionally further comprises isolating the amino acid sequence so produced.

35. Pharmaceutical composition comprising the amino acid sequence of claim 1, wherein said pharmaceutical composition is suitable for administration to a primate at interval(s) of at least 50% of the natural half-life of serum albumin in said primate.

36. The pharmaceutical composition according to claim 35 that further comprises at least one pharmaceutically acceptable carrier, diluent or excipient.

37.-39. (canceled)

40. Method of treatment, comprising administering the amino acid sequence according to claim 1 to a primate in need thereof, wherein said administration occurs at a frequency of at least 50% of the natural half-life of serum albumin in said primate.

41. Method according to claim 40, wherein the primate is human.

42. Method according to claim 41, wherein the medicament is administered at interval(s) of at least 7 days.

43. A method for extending or increasing the serum half-life of a therapeutic comprising
contacting the therapeutic with the amino acid sequence according to claim 1, such that the therapeutic is bound to or otherwise associated with the amino acid sequence.

44. The method of claim 43, wherein the therapeutic is a biological therapeutic.

45. The method of claim 44, wherein the biological therapeutic is a peptide or polypeptide, and wherein the step of contacting the therapeutic comprises preparing a fusion protein by linking the peptide or polypeptide with the amino acid sequence.

46. The method of claim 43, further comprising administering the therapeutic to a primate after the therapeutic is bound to or otherwise associated with the amino acid sequence.

47. The method of claim 46, wherein the serum half-life of the therapeutic in the primate is at least 1.5 times the half-life of therapeutic per se.

48. The method of claim 46, wherein the serum half-life of the therapeutic in the primate is increased by at least 1 hour compared to the half-life of therapeutic per se.


SEQUENCE LISTING (64 sequences)

SEQ ID NO: 1 - protein, 30 aa, Artificial sequence
  FRAMEWORK REGION 1; can be chemically synthesized
AVQLVESGGGLVQGGGSLRLACAASERIFD

SEQ ID NO: 2 - protein, 30 aa, Artificial sequence
  FRAMEWORK REGION 1; can be chemically synthesized
EVQLVESGGGLVQEGGSLRLACAASERIWD

SEQ ID NO: 3 - protein, 30 aa, Artificial sequence
  FRAMEWORK REGION 1; can be chemically synthesized
AVQLVESGGGLVQPGNSLRLSCAASGFTFR

SEQ ID NO: 4 - protein, 30 aa, Artificial sequence
  FRAMEWORK REGION 1; can be chemically synthesized
AVQLVDSGGGLVQPGGSLRLSCAASGFSFG

SEQ ID NO: 5 - protein, 30 aa, Artificial sequence
  FRAMEWORK REGION 1; can be chemically synthesized
AVQLVESGGGLVQPGGSLRLTCTASGFTFR

SEQ ID NO: 6 - protein, 30 aa, Artificial sequence
  FRAMEWORK REGION 1; can be chemically synthesized
QVQLAESGGGLVQPGGSLRLTCTASGFTFG

SEQ ID NO: 7 - protein, 30 aa, Artificial sequence
  FRAMEWORK REGION 1; can be chemically synthesized
QVQLVESGGGLVQPGGSLRLSCAASGFTFS

SEQ ID NO: 8 - protein, 5 aa, Artificial sequence
  COMPLEMENTARITY DETERMINING REGION 1; can be chemically synthesized
LNLMG

SEQ ID NO: 9 - protein, 5 aa, Artificial sequence
  COMPLEMENTARITY DETERMINING REGION 1; can be chemically synthesized
INLLG

SEQ ID NO: 10 - protein, 5 aa, Artificial sequence
  COMPLEMENTARITY DETERMINING REGION 1; can be chemically synthesized
SFGMS

SEQ ID NO: 11 - protein, 5 aa, Artificial sequence
  COMPLEMENTARITY DETERMINING REGION 1; can be chemically synthesized
SFGMS

SEQ ID NO: 12 - protein, 5 aa, Artificial sequence
  COMPLEMENTARITY DETERMINING REGION 1; can be chemically synthesized
SFGMS

SEQ ID NO: 13 - protein, 5 aa, Artificial sequence
  COMPLEMENTARITY DETERMINING REGION 1; can be chemically synthesized
SFGMS

SEQ ID NO: 14 - protein, 5 aa, Artificial sequence
  COMPLEMENTARITY DETERMINING REGION 1; can be chemically synthesized
NYWMY

SEQ ID NO: 15 - protein, 14 aa, Artificial sequence
  FRAMEWORK REGION 2; can be chemically synthesized
WYRQGPGNERELVA

SEQ ID NO: 16 - protein, 14 aa, Artificial sequence
  FRAMEWORK REGION 2; can be chemically synthesized
WYRQGPGNERELVA

SEQ ID NO: 17 - protein, 14 aa, Artificial sequence
  FRAMEWORK REGION 2; can be chemically synthesized
WVRQAPGKEPEWVS

SEQ ID NO: 18 - protein, 14 aa, Artificial sequence
  FRAMEWORK REGION 2; can be chemically synthesized
WVRQYPGKEPEWVS

SEQ ID NO: 19 - protein, 14 aa, Artificial sequence
  FRAMEWORK REGION 2; can be chemically synthesized
WVRQAPGKDQEWVS

SEQ ID NO: 20 - protein, 14 aa, Artificial sequence
  FRAMEWORK REGION 2; can be chemically synthesized
WVRQAPGEGLEWVS

SEQ ID NO: 21 - protein, 14 aa, Artificial sequence
  FRAMEWORK REGION 2; can be chemically synthesized
WVRVAPGKGLERIS

SEQ ID NO: 22 - protein, 17 aa, Artificial sequence
  COMPLEMENTARITY DETERMINING REGION 2; can be chemically synthesized
TCITVGDSTNYADSVKG

SEQ ID NO: 23 - protein, 16 aa, Artificial sequence
  COMPLEMENTARITY DETERMINING REGION 2; can be chemically synthesized
TITVGDSTSYADSVKG

SEQ ID NO: 24 - protein, 17 aa, Artificial sequence
  COMPLEMENTARITY DETERMINING REGION 2; can be chemically synthesized
SISGSGSDTLYADSVKG

SEQ ID NO: 25 - protein, 17 aa, Artificial sequence
  COMPLEMENTARITY DETERMINING REGION 2; can be chemically synthesized
SINGRGDDTRYADSVKG

SEQ ID NO: 26 - protein, 17 aa, Artificial sequence
  COMPLEMENTARITY DETERMINING REGION 2; can be chemically synthesized
AISADSSTKNYADSVKG

SEQ ID NO: 27 - protein, 17 aa, Artificial sequence
  COMPLEMENTARITY DETERMINING REGION 2; can be chemically synthesized
AISADSSDKRYADSVKG

SEQ ID NO: 28 - protein, 18 aa, Artificial sequence
  COMPLEMENTARITY DETERMINING REGION 2; can be chemically synthesized
RDISTGGGYSYYADSVKG

SEQ ID NO: 29 - protein, 32 aa, Artificial sequence
  FRAMEWORK REGION 3; can be chemically synthesized
RFTISMDYTKQTVYLHMNSLRPEDTGLYYCKI

SEQ ID NO: 30 - protein, 32 aa, Artificial sequence
  FRAMEWORK REGION 3; can be chemically synthesized
RFTISRDYDKNTLYLQMNSLRPEDTGLYYCKI

SEQ ID NO: 31 - protein, 32 aa, Artificial sequence
  FRAMEWORK REGION 3; can be chemically synthesized
RFTISRDNAKTTLYLQMNSLKPEDTAVYYCTI

SEQ ID NO: 32 - protein, 32 aa, Artificial sequence
  FRAMEWORK REGION 3; can be chemically synthesized
RFSISRDNAKNTLYLQMNSLKPEDTAEYYCTI

SEQ ID NO: 33 - protein, 32 aa, Artificial sequence
  FRAMEWORK REGION 3; can be chemically synthesized
RFTISRDNAKKMLYLEMNSLKPEDTAVYYCVI

SEQ ID NO: 34 - protein, 32 aa, Artificial sequence
  FRAMEWORK REGION 3; can be chemically synthesized
RFTISRDNAKKMLYLEMNSLKSEDTAVYYCVI

SEQ ID NO: 35 - protein, 32 aa, Artificial sequence
  FRAMEWORK REGION 3; can be chemically synthesized
RFTISRDNAKNTLYLQMNSLKPEDTALYYCAK

SEQ ID NO: 36 - protein, 8 aa, Artificial sequence
  COMPLEMENTARITY DETERMINING REGION 3; can be chemically synthesized
RRTWHSEL

SEQ ID NO: 37 - protein, 8 aa, Artificial sequence
  COMPLEMENTARITY DETERMINING REGION 3; can be chemically synthesized
RRTWHSEL

SEQ ID NO: 38 - protein, 6 aa, Artificial sequence
  COMPLEMENTARITY DETERMINING REGION 3; can be chemically synthesized
GGSLSR

SEQ ID NO: 39 - protein, 7 aa, Artificial sequence
  COMPLEMENTARITY DETERMINING REGION 3; can be chemically synthesized
GRSVSRS

SEQ ID NO: 40 - protein, 5 aa, Artificial sequence
  COMPLEMENTARITY DETERMINING REGION 3; can be chemically synthesized
GRGSP

SEQ ID NO: 41 - protein, 5 aa, Artificial sequence
  COMPLEMENTARITY DETERMINING REGION 3; can be chemically synthesized
GRGSP

SEQ ID NO: 42 - protein, 13 aa, Artificial sequence
  COMPLEMENTARITY DETERMINING REGION 3; can be chemically synthesized
DREAQVDTLDFDY

SEQ ID NO: 43 - protein, 11 aa, Artificial sequence
  FRAMEWORK REGION 4; can be chemically synthesized
WGQGTQVTVSS

SEQ ID NO: 44 - protein, 11 aa, Artificial sequence
  FRAMEWORK REGION 4; can be chemically synthesized
WGQGTQVTVSS

SEQ ID NO: 45 - protein, 11 aa, Artificial sequence
  FRAMEWORK REGION 4; can be chemically synthesized
SSQGTQVTVSS

SEQ ID NO: 46 - protein, 11 aa, Artificial sequence
  FRAMEWORK REGION 4; can be chemically synthesized
RTQGTQVTVSS

SEQ ID NO: 47 - protein, 11 aa, Artificial sequence
  FRAMEWORK REGION 4; can be chemically synthesized
SSPGTQVTVSS

SEQ ID NO: 48 - protein, 11 aa, Artificial sequence
  FRAMEWORK REGION 4; can be chemically synthesized
ASQGTQVTVSS

SEQ ID NO: 49 - protein, 11 aa, Artificial sequence
  FRAMEWORK REGION 4; can be chemically synthesized
RGQGTQVTVSS

SEQ ID NO: 50 - protein, 117 aa, Artificial sequence
  Nanobody sequence; can be chemically synthesized
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 51 - protein, 116 aa, Artificial sequence
  Nanobody sequence; can be chemically synthesized
EVQLVESGGGLVQEGGSLRLACAASERIWDINLLGWYRQGPGNERELVATITVGDSTSYADSVKGRFTISRDYDKNTLYLQMNSLRPEDTGLYYCKIRRTWHSELWGQGTQVTVSS

SEQ ID NO: 52 - protein, 115 aa, Artificial sequence [sequence omitted]

SEQ ID NO: 53 - protein, 116 aa, Artificial sequence
  Nanobody sequence; can be chemically synthesized
AVQLVDSGGGLVQPGGSLRLSCAASGFSFGSFGMSWVRQYPGKEPEWVSSINGRGDDTRYADSVKGRFSISRDNAKNTLYLQMNSLKPEDTAEYYCTIGRSVSRSRTQGTQVTVSS

SEQ ID NO: 54 - protein, 114 aa, Artificial sequence
  Nanobody sequence; can be chemically synthesized
AVQLVESGGGLVQPGGSLRLTCTASGFTFRSFGMSWVRQAPGKDQEWVSAISADSSTKNYADSVKGRFTISRDNAKKMLYLEMNSLKPEDTAVYYCVIGRGSPSSPGTQVTVSS

SEQ ID NO: 55 - protein, 114 aa, Artificial sequence
  Nanobody sequence; can be chemically synthesized
QVQLAESGGGLVQPGGSLRLTCTASGFTFGSFGMSWVRQAPGEGLEWVSAISADSSDKRYADSVKGRFTISRDNAKKMLYLEMNSLKSEDTAVYYCVIGRGSPASQGTQVTVSS

SEQ ID NO: 56 - protein, 123 aa, Artificial sequence
  Nanobody sequence; can be chemically synthesized
QVQLVESGGGLVQPGGSLRLSCAASGFTFSNYWMYWVRVAPGKGLERISRDISTGGGYSYYADSVKGRFTISRDNAKNTLYLQMNSLKPEDTALYYCAKDREAQVDTLDFDYRGQGTQVTVSS

SEQ ID NO: 57 - protein, 115 aa, Artificial sequence [sequence omitted]

SEQ ID NO: 58 - protein, 115 aa, Artificial sequence [sequence omitted]

SEQ ID NO: 59 - protein, 115 aa, Artificial sequence [sequence omitted]

SEQ ID NO: 60 - protein, 115 aa, Artificial sequence [sequence omitted]

SEQ ID NO: 61 - protein, 115 aa, Artificial sequence [sequence omitted]

SEQ ID NO: 62 - protein, 115 aa, Artificial sequence [sequence omitted]

SEQ ID NO: 63 - protein, 115 aa, Artificial sequence [sequence omitted]

SEQ ID NO: 64 - protein, 115 aa, Artificial sequence [sequence omitted]